(19) United States
(12) Patent Application Publication
(10) Pub. No.: US 2026/0028735 A1
(43) Pub. Date: Jan. 29, 2026

(54) METHODS AND SYSTEMS FOR POWER-TO-FUEL APPLICATIONS USING TAIL GAS FROM FUEL SYNTHESIS AND/OR METHANE IN HIGH TEMPERATURE ELECTROLYZER

(71) Applicant: Chevron U.S.A. Inc., San Ramon, CA (US)

(72) Inventors: Zihan Huang, Emeryville, CA (US); Lin Li, San Ramon, CA (US); Oliver Yair Gutiérrez-Tinoco, Martinez, CA (US); Tapan Das, Albany, CA (US); Evan Michael Visser, Spring, TX (US)

(21) Appl. No.: 19/277,586

(22) Filed: Jul. 23, 2025

Related U.S. Application Data
(60) Provisional application No. 63/674,985, filed on Jul. 24, 2024.

(51) Int. Cl. C25B 15/08; C07C 1/04; C07C 29/151; C07C 29/152; C25B 1/23

(52) U.S. Cl. CPC C25B 15/081; C07C 1/041; C07C 29/1518; C07C 29/152; C25B 1/23

(57) ABSTRACT

A continuous method includes passing a first steam feed stream and one or more of a recycled tail gas stream and a methane-rich feed stream to a cathode of a first electrolyzer containing the cathode, an anode and an electrolyte inserted between the cathode and the anode, thereby producing a cathode effluent including syngas, and passing the cathode effluent including syngas to a reactor unit, thereby producing a chemical product or a fuel-based product.

### PRIORITY CLAIM

The present application claims priority to U.S. Provisional Patent Application No. 63/674,985, entitled “Methods and Systems for Power-to-Fuel Applications Using Tail Gas from Fuel Synthesis and/or Methane in High Temperature Electrolyzer,” filed Jul. 24, 2024, the content of which is incorporated by reference herein in its entirety.

### BACKGROUND

Many energy production processes release carbon dioxide. In order to reduce the climate-damaging effects of carbon dioxide in the atmosphere, the carbon dioxide can be converted into other substances. However, this is made more difficult by the fact that carbon dioxide is stable and not very reactive. In addition, the carbon in the carbon dioxide molecule is in its highest oxidation state. Thus, carbon dioxide can no longer be used to generate energy by means of incineration.

Carbon dioxide can also be reduced to liquid hydrocarbons. For example, carbon dioxide can be directly hydrogenated to methane in the presence of a catalyst.

Producing a chemical using energy is also known as “Power-to-X” because energy (“power”) can be used to obtain a chemical (“X”). By using climate-damaging carbon dioxide as a starting material, this concept can contribute to reducing global warming.

### SUMMARY

In accordance with an illustrative embodiment, a continuous method comprises:
passing a first steam feed stream and one or more of a recycled tail gas stream and a methane-rich feed stream to a cathode of a first electrolyzer comprising the cathode, an anode and an electrolyte inserted between the cathode and the anode, thereby producing a cathode effluent comprising syngas, and
passing the cathode effluent comprising syngas to a reactor unit, thereby producing a chemical product or a fuel-based product.

In accordance with another illustrative embodiment, a system comprises:
a first electrolyzer comprising a cathode, an anode and an electrolyte inserted between the cathode and the anode, wherein the first electrolyzer is configured to receive in the cathode a first steam feed stream and one or more of a recycled tail gas stream from a reactor unit and a methane-rich feed stream, thereby producing a cathode effluent comprising syngas; and
a reactor unit configured to receive the cathode effluent comprising syngas, thereby producing a chemical product or a fuel-based product.

### BRIEF DESCRIPTION OF THE DRAWINGS

In combination with the accompanying drawings and with reference to the following detailed description, the features, advantages, and other aspects of the implementations of the present disclosure will become more apparent, and several implementations of the present disclosure are illustrated herein by way of example but not limitation. In the accompanying drawings:

FIG. 1A illustrates a method and system diagram scheme utilizing a high-temperature solid-oxide electrolyzer for steam electrolysis of a steam feed, and a recycled tail gas stream in the cathode to produce syngas via a reforming reaction for combining with carbon dioxide for sending to a downstream synthesis reactor for production of a chemical product or fuel-based product-based product, according to an illustrative embodiment.

FIG. 1B illustrates a method and system diagram scheme utilizing a high-temperature solid-oxide electrolyzer for steam electrolysis of a steam feed and a recycled tail gas stream and a methane-rich feed stream in the cathode to produce syngas via a reforming reaction for combining with carbon dioxide for sending to a downstream synthesis reactor for production of a chemical product or fuel-based product-based product, according to an illustrative embodiment.

FIG. 1C illustrates a method and system diagram scheme utilizing a high-temperature solid-oxide electrolyzer for steam electrolysis of a steam feed and a methane-rich feed stream in the cathode to produce syngas via a reforming reaction for combining with carbon dioxide for sending to a downstream synthesis reactor for production of a chemical product or fuel-based product-based product, according to an illustrative embodiment.

FIG. 2 is a schematic view showing a high-temperature solid-oxide electrolyzer (SOEC), according to an illustrative embodiment.

FIG. 3A illustrates a method and system diagram scheme utilizing a first high-temperature solid-oxide electrolyzer for steam electrolysis of a steam feed stream in the cathode to produce hydrogen and a second high-temperature solid-oxide electrolyzer for steam electrolysis of a steam feed and a recycled tail gas stream in the cathode to produce syngas via a reforming reaction for combining with carbon dioxide for sending to a downstream synthesis reactor for production of a chemical product or fuel-based product-based product, according to an illustrative embodiment.

FIG. 3B illustrates a method and system diagram scheme utilizing a first high-temperature solid-oxide electrolyzer for steam electrolysis of a steam feed stream in the cathode to produce hydrogen and a second high-temperature solid-oxide electrolyzer for steam electrolysis of a steam feed and a recycled tail gas stream and a methane-rich feed stream in the cathode to produce syngas via a reforming reaction for combining with carbon dioxide for sending to a downstream synthesis reactor for production of a chemical product or fuel-based product-based product, according to an illustrative embodiment.

FIG. 3C illustrates a method and system diagram scheme utilizing a first high-temperature solid-oxide electrolyzer for steam electrolysis of a steam feed stream in the cathode to produce hydrogen and a second high-temperature solid-oxide electrolyzer for steam electrolysis of a steam feed and a methane-rich feed stream in the cathode to produce syngas via a reforming reaction for combining with carbon dioxide for sending to a downstream synthesis reactor for production of a chemical product or fuel-based product-based product, according to an illustrative embodiment.

FIG. 4A illustrates a method and system diagram scheme utilizing a high-temperature solid-oxide electrolyzer for co-electrolysis of a steam and carbon dioxide feed stream, and a recycled tail gas stream in the cathode to produce syngas via a reforming reaction for sending to a downstream synthesis reactor for production of a chemical product or fuel-based product-based product, according to an illustrative embodiment.

FIG. 4B illustrates a method and system diagram scheme utilizing a high-temperature solid-oxide electrolyzer for co-electrolysis of a steam and carbon dioxide feed stream, and a recycled tail gas stream and a methane-rich feed stream in the cathode to produce syngas via a reforming reaction for sending to a downstream synthesis reactor for production of a chemical product or fuel-based product-based product, according to an illustrative embodiment.

FIG. 4C illustrates a method and system diagram scheme utilizing a high-temperature solid-oxide electrolyzer for co-electrolysis of a steam and carbon dioxide feed stream, and a methane-rich feed stream in the cathode to produce syngas via a reforming reaction for sending to a downstream synthesis reactor for production of a chemical product or fuel-based product-based product, according to an illustrative embodiment.

FIG. 5A illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for co-electrolysis of a steam and carbon dioxide feed stream, and a recycled tail gas stream in the cathode to produce syngas via a reforming reaction for sending to a downstream synthesis reactor for production of a chemical product or fuel-based product-based product, according to an illustrative embodiment.

FIG. 5B illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for co-electrolysis of a steam and carbon dioxide feed stream, and a recycled tail gas stream and a methane-rich feed stream in the cathode to produce syngas via a reforming reaction for sending to a downstream synthesis reactor for production of a chemical product or fuel-based product-based product, according to an illustrative embodiment.

FIG. 5C illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for co-electrolysis of a steam and carbon dioxide feed stream, and a methane-rich feed stream in the cathode to produce syngas via a reforming reaction for sending to a downstream synthesis reactor for production of a chemical product or fuel-based product-based product, according to an illustrative embodiment.

FIG. 6A illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for steam electrolysis of a steam feed and a recycled tail gas stream in the cathode to produce syngas via a reforming reaction for sending to a downstream synthesis reactor for production of a chemical product or fuel-based product-based product, according to an illustrative embodiment.

FIG. 6B illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for steam electrolysis of a steam feed and a recycled tail gas stream and a methane-rich feed stream in the cathode to produce syngas via a reforming reaction for sending to a downstream synthesis reactor for production of a chemical product or fuel-based product-based product, according to an illustrative embodiment.

FIG. 6C illustrates a method and system diagram scheme utilizing a first high-temperature solid-oxide electrolyzer for co-electrolysis of a steam and carbon dioxide feed stream in the cathode to produce syngas and a second high-temperature solid-oxide electrolyzer for steam electrolysis of a steam feed and a methane-rich feed stream in the cathode to produce syngas via a reforming reaction for sending to a downstream synthesis reactor for production of a chemical product or fuel-based product-based product, according to an illustrative embodiment.

### DETAILED DESCRIPTION

Various illustrative embodiments described herein are directed to methods and systems for the electrochemical conversion of steam or steam and carbon dioxide with one or more of a recycled tail gas stream from a reactor unit and a methane-rich feed stream into syngas in the cathode of an electrolyzer for the production of chemical products and/or fuel-based products.

“Power-to-gas,” “power-to-liquid,” and “power-to-fuel” (referred to as “Power-to-X” processes) represent promising approaches for bringing about a future conversion from fossil energy sources to an energy infrastructure which is based mainly on renewable energy sources (RES), for example wind power, solar power, geothermal energy, water power or hydroelectric power. Electricity-based or synthetic fuels are becoming ever more important, particularly in the transport sector or in industry. Such fuels, for example methane, methanol or derivatives or downstream products such as kerosene, gasoline, diesel, or other hydrocarbon-based products are produced, in particular, by synthesis from hydrogen and carbon dioxide.

Power-to-X processes utilize electrical energy to convert carbon dioxide into carbon-neutral fuels or chemicals. For example, it is possible to convert carbon dioxide and H₂ directly into multi-carbon species as desired fuels or chemicals. However, such a direct carbon conversion route for fuel synthesis is still at the early stage of development and faces significant technical challenges. On the other hand, the indirect route involves carbon dioxide and water being first converted into hydrogen and carbon monoxide by co-electrolysis, which can be further transformed into desired products in a downstream synthesis reaction process, e.g., an electrolyzer by way of co-electrolysis can convert water and carbon dioxide to syngas, and the produced syngas can be further reacted to make desired fuels or chemical products. For example, Fisher-Tropsch (FT) process can convert syngas to liquid fuels or base oil. In addition to the desired liquid hydrocarbon products, the FT process also produces a light end (fraction of C₁ to C₄ products) such as gaseous light products.

The gaseous light products, leaving the synthesis reactor as a tail gas, are less valuable than liquid products, more difficult to transport, and it is not practical to sell them as products for some applications. For conventional GTL (natural gas to liquid) plants, the tail gas is typically used as fuel gas. For CO₂ conversion through Power-to-X, it is undesirable to use tail gas as fuel. The burning of tail gas will produce CO₂, which will reduce the net CO₂ utilization rate (i.e., the percentage of total CO₂ feed converted to target liquid products). Therefore, there is a need to better utilize the tail gas while improving the overall energy efficiency and CO₂ utilization for a practicable CO₂ to a chemical product or fuel-based product process.

In the context of FT fuel synthesis, methane (CH₄) and other light hydrocarbons present in the tail gas of the fuel synthesis can be recycled back to the cathode of the SOEC. Steam Methane Reforming (SMR) can occur with steam feed in the cathode to produce syngas (hydrogen and carbon monoxide):

C_{X}H_{(2X+2)}+xH₂O→(2x+1)H₂+xCO

The high-temperature nature of the SOEC can enable the endothermic reaction required for SMR. Additionally, the large quantity of steam present in the cathode can potentially encourage SMR close to equilibrium conversion. This efficient use of heat and steam in the SOEC enhances the overall performance of the system. The hydrogen and carbon monoxide produced by SMR can then be used in the synthesis of fuels and chemicals, further reducing the overall energy consumption of the system.

Additionally, renewable natural gas (biomethane) can be used as a feedstock to the electrolyzer, and generate syngas needed for the downstream synthesis. By introducing methane, it is possible to reduce the electricity demand as previously mentioned while maintaining a consistent hydrogen to carbon ratio of the syngas product, as preferred for downstream synthesis. This is especially attractive at times when renewable electricity is less available, significantly increasing the overall flexibility of the system and the diversification of feedstock. Methanol synthesis from CO₂ is an attractive option for CO₂ utilization because methanol is liquid fuel that is a relatively easy to transport with numerous applications. Although direct CO₂ and hydrogen to methanol conversion technology is available, the slower reaction kinetics result in a significantly lower per-pass carbon conversion and therefore much larger reactors are required. By incorporating SOEC co-electrolysis the resulting syngas product allows for traditional methanol synthesis, and provides a destination for any light hydrocarbon by-products.

Accordingly, the non-limiting illustrative embodiments disclosed herein overcome the foregoing drawbacks by utilizing the recycling of a tail gas stream from downstream fuel synthesis and/or a methane-rich feed stream to an SOEC electrolysis to enhance the overall energy efficiency and carbon dioxide utilization rate for converting carbon dioxide to chemical products and/or fuel-based products. The non-limiting illustrative embodiments described herein are able to maximize the utilization of the available heat from the recycled tail gas stream and/or methane-rich feed stream being sent to the electrolyzer, thereby reducing the electrolyzer electricity consumption while improving energy efficiency and reducing electricity cost.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and methods are described in terms of “comprising” various components or steps, the systems and methods can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “continuous” as used herein shall be understood to mean a system that operates without interruption or cessation for a period of time, such as where at least reactant(s) are continually fed into a reaction zone of a reactor unit and products are continually or regularly withdrawn without stopping the reaction in the reaction zone.

The term “reactor unit product-forming feed stream” as used herein shall be understood to mean a feed stream which contains hydrogen and a sufficient carbon amount. For example, for Fischer-Tropsch (FT) synthesis via syngas, the optimal ratio of hydrogen (H₂) to carbon monoxide (CO) is approximately 3:1. However, for direct hydrogenation in FT synthesis, where hydrogen (H₂) reacts with carbon dioxide (CO₂), the optimal ratio is 4:1. These ratios can achieve efficient and effective FT synthesis such that when passed into the reactor unit one or more of a chemical product and/or fuel can be produced as discussed below. As one skilled in the art will understand, the hydrogen can be formed from one or more of steam electrolysis of a steam feed stream in one or more electrolyzers described herein, co-electrolysis of a steam and carbon dioxide feed stream in one or more electrolyzers, and a reforming reaction of a steam feed stream with one or more of a recycled tail gas stream from a reactor unit and a methane-rich feed stream in one or more electrolyzers. The carbon monoxide can be formed from one or more of co-electrolysis of a steam and carbon dioxide feed stream in one or more electrolyzers, and a reforming reaction of a steam feed stream with one or more of a recycled tail gas stream from a reactor unit and a methane-rich feed stream in one or more electrolyzers.

The term “electrode” is meant, in the sense of the present disclosure, an electronic conductor capable of capturing or releasing electrons. An oxidation reaction occurs at the anode, whereas a reduction reaction occurs at the cathode.

The term “electrolysis” is a technique that uses direct electric current to drive an otherwise non-spontaneous chemical reaction. For example, the electrolysis of water is the process of using electricity to electrochemically decompose water into oxygen and hydrogen. The term “electrolyzer,” also called an “electrolysis device,” refers to a unit where this chemical reaction may take place.

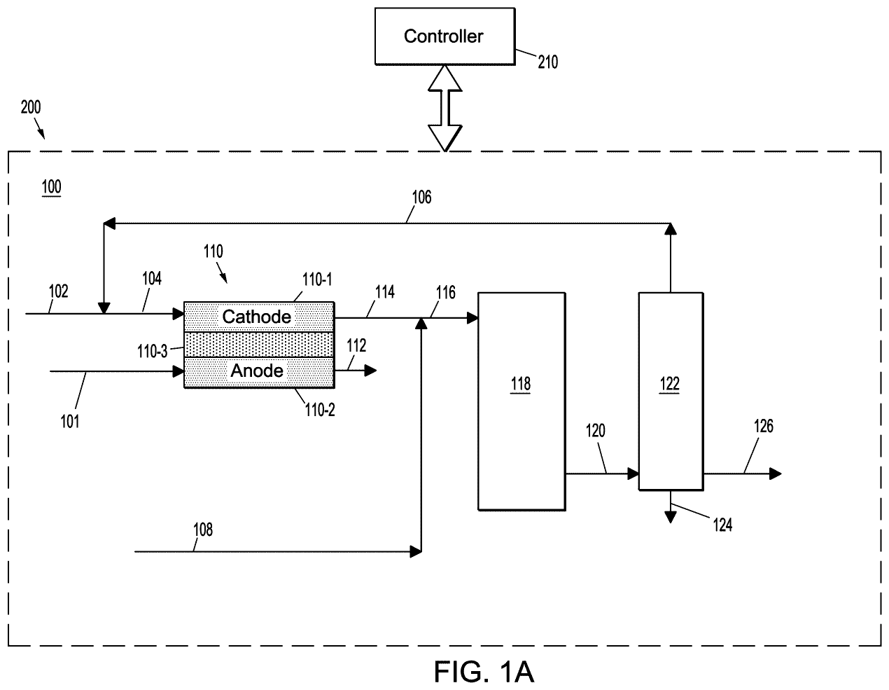

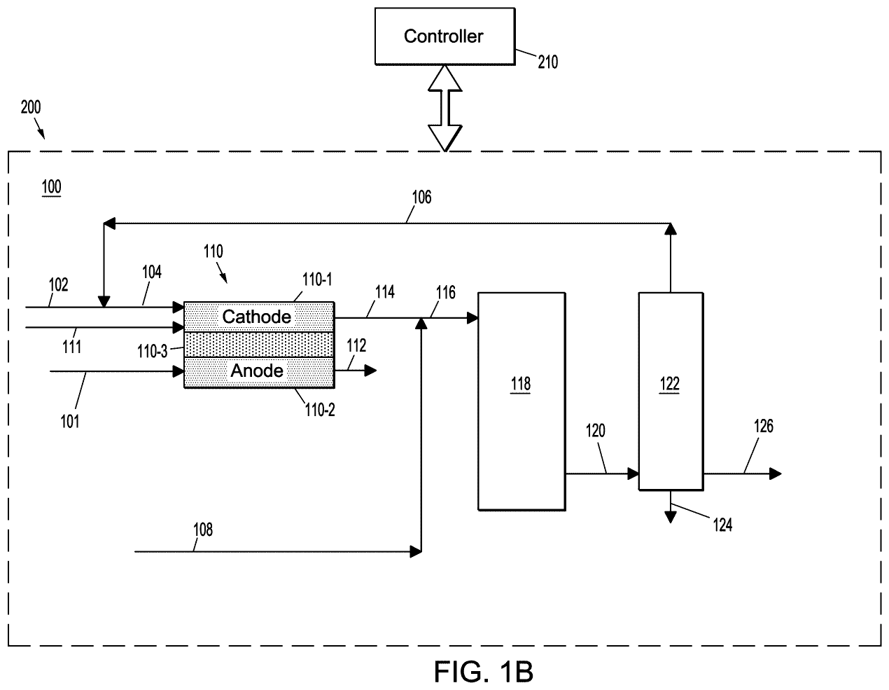

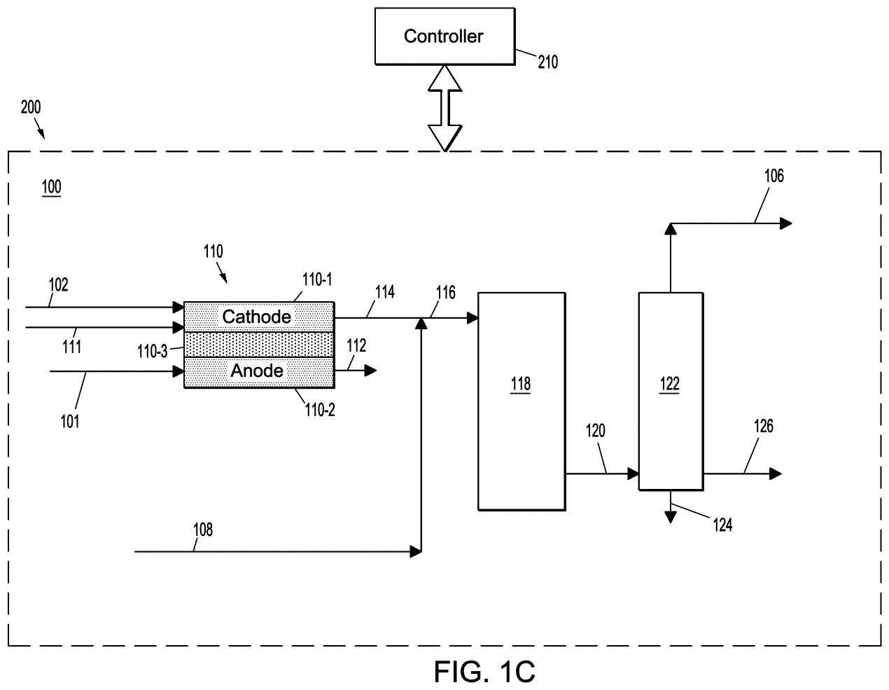

The illustrative embodiments of the present disclosure will be specifically described below with reference to the accompanying drawings. For the purpose of clarity, some steps leading up to the production of syngas through electrochemical conversion of steam or steam and carbon dioxide in a cathode of an electrolyzer with one or more of a recycled tail gas stream from a reactor unit and a methane-rich feed stream to produce chemical products and/or fuel-based products as illustrated in FIGS. 1A-6C are omitted. In other words, one or more well-known processing steps which are not illustrated but are well-known to those of ordinary skill in the art have not been included in the figures. This is not intended to be interpreted as a limitation of any particular embodiment, or illustration, or scope of the claims.

FIGS. 1A-1C illustrate a method and system diagram scheme utilizing a high-temperature solid-oxide electrolyzer for steam electrolysis of a steam feed and one or more of a recycled tail gas stream from a reactor unit and a methane-rich feed stream in the cathode to produce syngas via a reforming reaction.

Referring now to FIGS. 1A-1C, a system 100 includes an electrolyzer 110 comprising a cathode 110-1, an anode 110-2 and an electrolyte 110-3 inserted between cathode 110-1 and anode 110-2. In some embodiments, as depicted in FIG. 1A, electrolyzer 110 receives a cathode feed stream 104 formed from a steam feed stream 102 and a recycled tail gas stream 106 from a separation unit 122 as discussed below into cathode 110-1 and an anode purge stream 101 into anode 110-2. In some embodiments, as depicted in FIG. 1B, electrolyzer 110 receives cathode feed stream 104 formed from steam feed stream 102 and recycled tail gas stream 106 from separation unit 122, and a methane-rich feed stream 111 into cathode 110-1 and anode purge stream 101 into anode 110-2. In some embodiments, as depicted in FIG. 1C, electrolyzer 110 receives steam feed stream 102 and methane-rich feed stream 111 into cathode 110-1 and anode purge stream 101 into anode 110-2.

Although steam feed stream 102 and recycled tail gas stream 106 are shown entering cathode 110-1 as cathode feed stream 104, this is merely illustrative and steam feed stream 102 and recycled tail gas stream 106 can each enter cathode 110-1 as a single stream or can be combined with methane-rich feed stream 111, when applicable, in any type of configuration. For example, steam feed stream 102 and methane-rich feed stream 111 can be combined as a single stream when entering cathode 110-1, or recycled tail gas stream 106 and methane-rich feed stream 111 can be combined as a single stream when entering cathode 110-1, or steam feed stream 102, recycled tail gas stream 106 and methane-rich feed stream 111 can be combined as a single stream when entering cathode 110-1. Anode purge stream 101 includes one or more of air, carbon dioxide or an inert gas such as N₂. Steam feed stream 102 can be obtained from any means known in the art.

In some embodiments, anode purge stream 101 such as air may be pressurized to produce a pressurized anode purge stream using fans, blowers, compressors or a combination thereof (not shown). The air compressor may be centrifugal, mixed-flow, axial-flow, reciprocating, rotary screw, rotary vane, scroll, diaphragm compressor, or a combination thereof. In some embodiments, the pressurized anode purge stream may have a pressure between about 1 bar to about 20 bar. In some embodiments, the pressurized anode purge stream may have a pressure of about 1 bar, about 1.2 bar, about 5 bar, about 10 bar, about 15 bar, or about 20 bar. In some embodiments, the pressurized anode purge stream may have a pressure of from about 1 bar to about 3 bar, e.g., about 1 bar, about 1.2 bar, about 1.4 bar, about 1.6 bar, about 1.8 bar, about 2.0 bar, about 2.2 bar, about 2.4 bar, about 2.6 bar, about 2.8 bar or about 3 bar.

Anode purge stream 101 serves as a purge gas to carry oxygen generated at anode 110-2 to produce an anode effluent 112 composed of at least oxygen, for example, less than 50% of oxygen. Anode effluent 112 can be discarded or recycled back to electrolyzer 110. In an illustrative embodiment, electrolyzer 110 can be any suitable high temperature electrolyzer comprising cathode 110-1, anode 110-2 and electrolyte 110-3 inserted between cathode 110-1 and anode 110-2. In a non-limiting illustrative embodiment, electrolyzer 110 is a high temperature solid oxide electrolyzer (also referred to as SOEC) for steam electrolysis comprising:
a first porous conductive electrode, or “cathode,” to be supplied with steam for the production of dihydrogen,
a second porous conductive electrode, or “anode,” via which the dioxygen (O₂) produced by the electrolysis of the water injected onto the cathode escapes, and
a solid oxide membrane (dense electrolyte) sandwiched between the cathode and the anode, the membrane being anionically conductive at high temperatures, usually temperatures above about 700° C. and up to about 950° C.

Electrolyzer 110 may receive input energy (i.e., electricity) from an intermittent source such as solar power (including photovoltaic and reflective), wind power, tidal power, wave power, batteries, and other intermittent energy sources known in the art and combinations thereof. Alternatively, or in addition, electrolyzer 110 may receive input energy from a non-intermittent source, such as an electricity grid (e.g., a regional electricity grid, a municipal electricity grid, or a microgrid), natural gas, coal, nuclear, and other non-intermittent sources known in the art and combinations thereof. Electrolyzer 110 may therefore be electricity connectable to an intermittent energy input, a non-intermittent source, or a combination thereof. In particular embodiments, electrolyzer 110 may receive input energy from the photovoltaic panel.

In some embodiments, electrolyzer 110 may be receiving electricity from a photovoltaic panel. At night, electrolyzer 110 may be operated in “hot standby” mode to conserve electricity, or electrolyzer 110 may be electricity connected to another power source to continue operating at night. In particular, electrolyzer 110 may be connected to a power grid such as a regional power grid, a municipal power grid, or a micro grid, and electrolyzer 110 may run when the price of electricity is low.

System 100 may further comprise an energy storage mechanism or a plurality of energy storage mechanisms. The energy storage mechanism may comprise any mechanism or apparatus operable to store energy such as electricity, thermal energy, etc. For example, the energy storage mechanism may include batteries (e.g., lead-acid batteries, lithium-ion batteries, lithium iron batteries, etc.), water, flywheels, compressed air, pumped hydroelectric, or other energy storage mechanisms known in the art and combinations thereof.

As an overview, for high-temperature steam electrolysis (HTSE), steam (H₂O) is injected into at least the cathode compartment of the electrolyzer. Under the effect of the electrical current applied to the cell, the dissociation of water molecules in the form of steam occurs at the interface between the hydrogen electrode (cathode) and the electrolyte, where this dissociation produces dihydrogen gas (H₂) and oxygen ions (O²⁻). Dihydrogen (H₂) is collected and discharged at the outlet of the hydrogen compartment. The oxygen ions (O²⁻) migrate through the electrolyte and form dioxygen (O₂) at the interface between the electrolyte and the oxygen electrode (anode). A draining gas, such as air, can circulate at the anode and thus collect the oxygen generated in gas form at the anode.

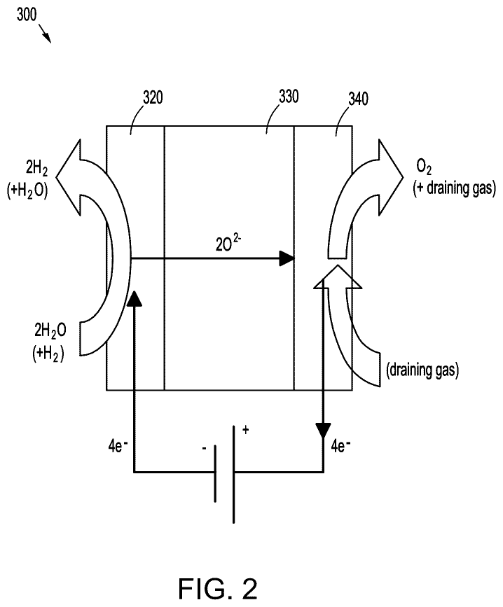

In an illustrative embodiment, FIG. 2 shows a schematic view of the principle of operation of a high-temperature solid-oxide electrolyzer (SOEC). Such an electrolyzer is an electrochemical device for producing hydrogen (and oxygen) under the effect of an electrical current. In these electrolyzers, the high-temperature electrolysis of water is performed using steam. Thus, the function of such an electrolyzer is to transform the steam into hydrogen and oxygen according to the following chemical reaction:

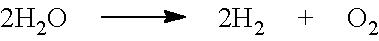

This reaction occurs electrochemically in the cells of the electrolyzer. As schematically shown in FIG. 2, each basic electrolysis cell 300 is formed by a cathode 320 and an anode 340, placed on either side of an electrolyte 330. The two electrodes (i.e., cathode 320 and anode 340) are electronic and/or ionic conductors, made of porous material, and electrolyte 330 is impervious to gas, an electronic insulator and an ion conductor. Electrolyte 330 may in particular be an anion conductor, and more specifically an anion conductor of O²⁻ ions, and the electrolyzer is then referred to as an anion electrolyzer, by contrast with proton electrolytes (H⁺).

The electrochemical reactions occur at the interface between each of the electronic conductors and the ion conductor.

At cathode 320, the half-reaction is as follows:

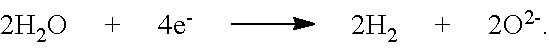

At the anode 340, the half-reaction is as follows:

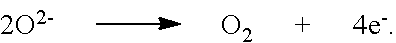

Electrolyte 330, inserted between the two electrodes, i.e., cathode 320 and anode 340, is the site of migration of the O²⁻ ions under the effect of the electrical field created by the difference in potential imposed between anode 340 and cathode 320.

As indicated between parentheses in FIG. 2, the steam at the cathode inlet can be accompanied by hydrogen H₂ and the hydrogen produced and recovered at the outlet can be accompanied by steam. Similarly, a draining gas, such as air, may also be injected at the inlet to discharge the oxygen produced. The injection of a draining gas has the additional function of acting as a temperature controller.

A basic electrolyzer, or electrolysis reactor, therefore consists of a basic cell as described above, with cathode 320, electrolyte 330, and anode 340, and two monopolar connectors, which provide electrical, hydraulic and thermal distribution functions.

To increase the flow rates of hydrogen and oxygen produced, a stack of a plurality of basic electrolysis cells one on top of another can be used, separating them with interconnection devices, usually called interconnectors or bipolar interconnection plates. The assembly is positioned between two end interconnection plates that support the electrical and gas supplies of the electrolyzer (electrolysis reactor).

A high-temperature solid-oxide electrolyzer (SOEC) thus comprises at least one, and generally a plurality of electrolysis cells stacked one on top of another, each basic cell being formed by an electrolyte, a cathode and an anode, the electrolyte being inserted between the cathode and the anode.

Thus, the function of a so-called cathode compartment is to distribute the electrical current and steam as well as to recover hydrogen at the cathode in contact.

The function of a so-called anode compartment is to distribute the electrical current and to recover oxygen at the anode in contact, optionally by means of a draining gas.

In some embodiments, as depicted in FIG. 1A, electrolyzer 110 receives steam feed stream 102 and recycled tail gas stream 106 containing methane and light hydrocarbons (e.g., CₓH₍₂ₓ₊₂₎ such as C₂ to C₄) as cathode feed stream 104 into cathode 110-1. Recycled tail gas stream 106 can reach a temperature of about 750° C. to about 850° C. after heat integration with electrolyzer 110. For example, recycled tail gas stream 106 can be passed through one or more heat exchangers (not shown) to increase its temperature to a temperature of about 750° C. to about 850° C. for heat integration with electrolyzer 110.

In some embodiments, as depicted in FIG. 1B, electrolyzer 110 receives steam feed stream 102, recycled tail gas stream 106 containing methane and light hydrocarbons (e.g., CₓH₍₂ₓ₊₂₎ such as C₂ to C₄) into cathode 110-1 and methane-rich feed stream 111 containing methane and light hydrocarbons (e.g., CₓH₍₂ₓ₊₂₎ such as C₂ to C₄). In some embodiments, methane-rich feed stream 111 can be derived from a light hydrocarbon feed stream comprising methane or natural gas or renewable natural gas such as, for example, a light hydrocarbon feed stream comprising greater than about 50%, or greater than about 80%, or greater than about 90%, or greater than about 95%, or greater than about 99% methane. As used herein, natural gas or renewable natural gas comprises methane and potentially higher alkanes, carbon dioxide, nitrogen or other gases, and/or sulfur-containing compounds such as hydrogen sulfide, and mixtures thereof.

In some embodiments, as depicted in FIG. 1C, electrolyzer 110 receives steam feed stream 102 and methane-rich feed stream 111 into cathode 110-1. FIG. 1C further shows recycled tail gas stream 106 exiting separation unit 122 where it can be further processed or reused as, for example, a fuel gas.

The methane and the other light hydrocarbon present in recycled tail gas stream 106 and/or methane-rich feed stream 111 react in cathode 110-1 with steam feed stream 102 via a reforming reaction such as steam methane reforming, together with steam electrolysis in cathode 110-1, to produce a cathode effluent 114 having a temperature of about 750° C. to about 850° C. and composed of carbon monoxide (CO), hydrogen (H₂), water (H₂O) and carbon dioxide (CO₂). For example, in some embodiments, cathode 110-1 of electrolyzer 110 can contain nickel which is an effective catalyst for a steam reforming reaction. Accordingly, recycled tail gas stream 106 and/or methane-rich feed stream 111 can be converted into syngas, i.e., H₂ and CO.

In some embodiments, electrolyzer 110 may operate at a temperature of about 700° C. to about 950° C. In some embodiments, electrolyzer 110 may operate at a temperature of about 700° C., about 720° C., about 730° C., about 740° C., about 750° C., about 760° C., about 770° C., about 780° C., about 790° C., about 800° C., about 810° C., about 820° C., about 830° C., about 840° C., about 860° C., about 880° C., about 900° C., about 910° C., about 920° C., about 930° C., about 940° C., or about 950° C., where any range from these limits are contemplated herein. In some embodiments, cathode 110-1 may operate at a temperature between about 750° C. to about 850° C., and anode 110-2 may operate at a temperature between about 750° C. to about 850° C.

In some embodiments, electrolyzer 110 may operate at a pressure between about 1 bar to about 20 bars. In some embodiments, electrolyzer 110 may operate at a pressure of about 1.02 bar, about 3 bar, about 5 bar, about 7 bar, about 9 bar, about 10 bar, about 15 bar or about 20 bar, where any range from these limits are contemplated herein. In some embodiments, the SOEC may operate at a pressure of about 1 bar to about 3 bar, e.g., about 1 bar, about 1.2 bar, about 1.4 bar, about 1.6 bar, about 1.8 bar, about 2.0 bar, about 2.2 bar, about 2.4 bar, about 2.6 bar, about 2.8 bar or about 3 bar, where any range from these limits are contemplated herein.

The material of the solid oxide electrolyzer electrodes (i.e., cathode 110-1 and anode 110-2) may be based on ceramic materials that exhibit stability through reduction-oxidation (redox) cycles, electrocatalytic activity, and mixed ionic and electronic conductivity in reducing atmospheres. The material of the solid oxide electrodes may be metal or metal oxide-based material (e.g., Ni-based electrodes). In some embodiments, the cathode and anode may be constructed of any suitable material including, for example, (La,Sr)(Fe,Co)O₃(LSCF), (Sm,Sr)CoO₃, and Sr-doped LaMnO₃ for the anode electrode (anode) and Ni—YSZ, Ni—ScSZ, La₂NiO₄, and Ni—ZrO₂ for the cathode electrode. Electrode support materials and functional layers include nickel cermets, and other electronic conductors such as (Sr_{0.8}La_{0.2})TiO₃(SLT). The electrolyte may be comprised of any suitable material such as, for example, yttria-stabilized zirconia (YSZ), (La_{0.6}Sr_{0.4})(Ga_{0.8}Mg_{0.2})O₃ (LSGM), Sc-stabilized zirconia (SSZ), and doped ceria. A SOEC cell architecture includes both electrode- and electrolyte-supported cell constructions and ceramic or metallic interconnects. It is to be understood that the above materials are merely exemplary and any known materials for use in the SOEC cell architecture are contemplated.

Cathode effluent 114 (syngas (CO and H₂), H₂O and CO₂) having a temperature of about 750° C. to about 850° C. can be sent to one or more heat exchangers (not shown) to remove heat from cathode effluent 114 before being combined with a carbon dioxide feed stream 108 to form a reactor feed stream 116 composed of carbon monoxide (CO), hydrogen (H₂), minimal water (H₂O), if performing dehydration step, and carbon dioxide (CO₂) and having a temperature of about 200° C. to about 300° C. Carbon dioxide feed stream 108 enters system 100 at or around a temperature of about 20° C. However, after undergoing heat integration with the fuel synthesis effluent stream (i.e., a stream 120), the temperature of the carbon dioxide can increase to a range of about 200° C. to about 300° C.

System 100 further includes a reactor unit 118 for receiving reactor feed stream 116. In some embodiments, reactor unit 118 can be one or more of a Fischer-Tropsch reactor, a methanol reactor and/or a dimethyl ether reactor and a hydrogenation reactor. In some embodiments, reactor unit 118 can be a Fischer-Tropsch reactor for processing reactor feed stream 116. In some embodiments, a Fischer-Tropsch reactor is utilized for processing reactor feed stream 116 thereby converting syngas to a Fischer-Tropsch product by conventional techniques. For example, in a Fischer-Tropsch reaction, syngas composed of carbon monoxide (CO) and hydrogen gas (H₂), is converted in the presence of a Fischer-Tropsch catalyst (e.g., iron- or cobalt-based catalyst) into hydrocarbon products, water and other byproducts. For example, the Fischer-Tropsch process involves a series of chemical reactions for converting syngas to a variety of hydrocarbons, ideally having the formula (CₙH₂ₙ₊₂). In an illustrative embodiment, the more useful reactions produce alkanes such as follows: (2n+1) H₂+n CO→CₙH₂ₙ₊₂+nH₂O where n is from 1 to 70. In addition to the desired liquid hydrocarbon products, a Fisher-Tropsch reaction can also produce light ends (fraction of C₁ to C₄ products). In a non-limiting illustrative embodiment, Table 1 shows a product yield for Fisher-Tropsch reaction using syngas as feed.

TABLE 1

| Products | Selectivity % |
|---|---|
| Gas (C₁ to C₄) | 5 to 7 |
| Naphtha (C₅ to C₇) | 10 to 15 |
| Diesel | 30 to 35 |
| Base Oil | 45 to 50 |

The products produced from the reaction process in reactor unit 118 can then be discharged from reactor unit 118 as a reactor synthesis effluent 120 comprising Fischer-Tropsch products.

In some embodiments, reactor unit 118 can be a methanol reactor and/or dimethyl ether reactor, rather than a Fischer-Tropsch reactor for processing reactor feed stream 116. For example, reactor unit 118 can be a synthesis reactor for converting CO and hydrogen in reactor feed stream 116 to such desired products as methanol which can thereafter be converted to, for example, dimethyl ether, in the same or separate reactor (not shown) by well-known conventional techniques, e.g., by methanol synthesis and in-situ dehydration, in which the in-situ methanol conversion can alleviate the thermodynamic limits of methanol synthesis, resulting in higher dimethyl ether yield.

In a non-limiting illustrative embodiment, a methanol synthesis section can be as represented below.

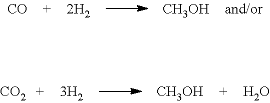

The products produced from the reaction process in reactor unit 118 can then be discharged from reactor unit 118 as reactor synthesis effluent 120 comprising one or more of methanol and/or dimethyl ether.

Numerous types of reactor systems have been developed for carrying out the Fischer-Tropsch or methanol/dimethyl ether reaction. For example, Fischer-Tropsch or methanol/dimethyl ether reactor systems include fixed bed reactors, especially multi-tubular fixed bed reactors, fluidized bed reactors, such as entrained fluidized bed reactors, and slurry bed reactors such as three-phase slurry bubble columns and ebullated bed reactors. The present disclosure is applicable to all types of reactor systems. The reactors each have an inlet for receiving synthesis gas and an outlet for discharging an effluent stream.

In this particular embodiment, system 100 can further include a reverse water gas shift (RWGS) reactor unit (not shown) for receiving reactor feed stream 116 in the case where reactor unit 118 is a Fischer-Tropsch reactor or a methanol reactor. The reverse water gas shift reaction converts CO₂ and H₂ into CO and H₂O. For example, catalyst and/or reactor configurations for performing Fischer-Tropsch synthesis or methanol synthesis can readily use CO as a reactant but typically cannot use carbon dioxide (CO₂). As a result, performing the reverse water gas shift reaction can allow CO₂ and H₂ to be used to form syngas, which can then be used for a fuel synthesis reaction such as methanol synthesis.

The reverse water gas shift reaction is part of the same equilibrium as the water gas shift reaction. In that equilibrium, formation of CO and H₂O is favored by increased temperatures. In particular, due to a competing equilibrium reaction for formation of methane, the equilibrium conversion of CO₂ passes through a minimum at roughly 600° C. By performing the reverse water gas shift reaction at temperatures of 700° C. or more, or 800° C. or more, or 900° C. or more (such as up to 1600° C. or possibly still higher), the equilibrium conversion of CO₂ can be increased while operating at temperatures with relatively fast kinetics. In various aspects, the amount of CO₂ in the reaction products can be about 0.5 vol % to about 5.0 vol %, or about 0.5 vol % to about 3.0 vol %, or about 0.5 vol % to about 2.5 vol %, or about 1.0 vol % to about 5.0 vol %, or about 1.0 vol % to about 3.0 vol %, or about 1.0 vol % to about 2.5 vol %.

The RWGS reactor unit can be a cylindrical vessel (e.g., with a length longer than diameter). The entrance to the reactor vessel can be smaller than the overall diameter of the vessel. The reactor vessel can be a steel vessel that is lined with an inert material that is non-reactive with the heated syngas. The steel vessel can be insulated to limit heat loss. Various types of insulation include poured or castable refractory lining or insulating bricks may be used to limit the heat losses to the environment.

A bed of catalyst can be inside the RWGS reactor unit. The catalyst can be in the form of granules, pellets, spheres, trilobes, quadra-lobes, monoliths, or any other engineered shape (e.g., to minimize pressure drop across the reactor). The shape and particle size of the catalyst particles can be managed such that pressure drop across the reactor is less than 50 pounds per square inch (psi) (e.g., between 10 psi and 50 psi), and in some cases, less than 20 psi (e.g., between 10 psi and 20 psi). The size of the catalyst form can have a characteristic dimension of between 1 mm to 10 mm. The catalyst particle can be a porous material with an internal surface area greater than about 80 m²/g (e.g., between about 80 m²/g and up to about 120 m²/g).

In illustrative embodiments, the RWGS catalyst is a solid solution catalyst that primarily comprises Ni₂Mg impregnated on a high-temperature spinel. This high-performance, solid-solution, Ni-based catalyst can be highly versatile and perform the RWGS reaction efficiently.

In one aspect, the RWGS reactor unit comprises one or more RWGS reactor units, arranged in series e.g., two or more RWGS reactor units. Each of the RWGS reactor units may be either adiabatic or a heated reactor. Heating can be achieved by means of heat integration with the effluent of fuel synthesis reaction.

The reverse water gas shift effluent generated from reactor feed stream 116 is a stream comprising CO, H₂O, unreacted CO₂, and H₂. The reverse water gas shift effluent can have a temperature from about 400° C. to about 900° C., depending on the extent of reverse water gas shift reaction and extent of heating. In an illustrative embodiment, the reverse water gas shift effluent can be dehydrated before sending to reactor unit 118.

In some embodiments, reactor unit 118 can be a so-called direct hydrogenation reactor unit where CO₂ in reactor feed stream 116 can be directly hydrogenated into liquid hydrocarbon products such as, for example, methanol, or Fischer-Tropsch products, as known in the art. For example, in non-limiting illustrative embodiments, reactor unit 118 can be a synthesis reactor for converting carbon dioxide and hydrogen to desired products such as methanol which can thereafter be converted to, for example, dimethyl ether, in the same or separate reactor (not shown) by conventional techniques, e.g., by methanol synthesis and in-situ dehydration, in which the in-situ methanol conversion can alleviate the thermodynamic limits of methanol synthesis, resulting in higher dimethyl ether yield.

In a non-limiting illustrative embodiment, Table 2 shows a product yield for a direct hydrogenation process reaction of carbon dioxide and hydrogen.

TABLE 2

| Products | Selectivity % |
|---|---|
| Gas (C₁ to C₄) | 10 to 15 |
| Naphtha (C₅ to C₇) | 15 to 20 |
| Jet Fuel (C₈ to C₁₆) | 55 to 65 |
| Diesel (C₁₇ to C₂₃) | 5 to 10 |

The products produced from the reaction process can then be discharged from reactor unit 118 as reactor synthesis effluent 120.

Accordingly, for purposes of this illustrative embodiment, reactor synthesis effluent 120 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of carbon dioxide and hydrogen is contemplated herein for use as reactor synthesis effluent 120.

The Fischer-Tropsch products, methanol/dimethyl ether products and/or direct hydrogenation products can then be discharged from reactor unit 118 as reactor synthesis effluent 120 and sent to separation unit 122. The terms “separation unit” and “separator” refer to any separation device(s) that at least partially separates one or more chemical constituents in a mixture from one another. For example, a separation unit may selectively separate different chemical species from one another, forming one or more chemical fractions. Examples of separation units include, without limitation, distillation columns, fractionators, flash drums, knock-out drums, knock-out pots, centrifuges, filtration devices, traps, scrubbers, expansion devices, membranes, solvent extraction devices, high-pressure separators, low-pressure separators, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. As used in this disclosure, one or more chemical constituents may be “separated” from a process stream to form a new process stream. Generally, a process stream may enter a separation unit and be divided or separated into two or more process streams of desired composition.

The Fischer-Tropsch products, methanol/dimethyl ether products and/or direct hydrogenation products in reactor synthesis effluent 120 can be separated and collected following conventional fractional distillation to produce product effluent 126 for sending further downstream for processing, while a portion of any unreacted syngas can be recycled to reactor unit 118 for further conversion (not shown), and any methane and light hydrocarbons such as C₁ to C₄ light ends produced from the synthesis reactions can be recycled from separation unit 122 as recycled tail gas stream 106 back to cathode 110-1 of electrolyzer 110 (see FIGS. 1A and 1B) or sent for further processing or reused as, for example, fuel gas (see FIG. 1C) as discussed above. A liquid stream 124 including at least hydrocarbons and water can be sent for further downstream processing as known in the art. For example, product effluent 126 can be sent for further downstream processing for creating high value liquid fuels, such as gasoline, diesel, and jet, or base oils.

In some embodiments, system 100 may include a hydrocracker unit and/or fractionation unit (not shown) to upgrade product effluent 126. For example, the hydrocracker unit can employ a high temperature, high pressure catalytic process that can upgrade heavy Fischer-Tropsch liquid (HFTL) and medium Fischer-Tropsch liquid (MFTL) hydrocarbon streams into a transportation fuel or a blending component meeting chemical and physical properties.

Accordingly, when reactor synthesis effluent 120 comprises Fischer-Tropsch products, the Fischer-Tropsch products can be separated out in separation unit 122 as a product effluent 126 and sent for further downstream processing as known in the art. For example, product effluent 126 can be sent for further downstream processing for creating high value liquid fuels, such as gasoline, diesel, and jet.

In one or more illustrative embodiments, a system processing environment 200 comprises each of the components of system 100 described herein, as well as a controller 210 operatively coupled to system 100. Controller 210 is configured to control operations of one or more of the components of system 100 discussed above. In one illustrative embodiment, controller 210 is configured to actuate one or more of the functionalities of system 100 described herein. For example, controller 210 can comprise one or more processing devices configured to load software instructions from one or more memory devices and execute the software instructions to generate data and/or control signals that can be applied to one or more components of system 100 so as to actuate the functionalities described herein. Actuation of the components by the data and/or control signals may be affected electrically, electromechanically, electrochemically, and/or the like, depending on the nature of the specific component of system 100 being actuated.

Thus, in some embodiments, controller 210 comprises a combination of hardware and software components. For example, the one or more processing devices of controller 210 may comprise one or more microprocessors, one or more microcontrollers, one or more application-specific devices, or other types of processing circuitry, as well as portions or combinations thereof. Further, the one or more memory devices of controller 210 may comprise random access memory (RAM), read-only memory (ROM), or other types of memory, in any combination. It is to be appreciated that the specific architecture of controller 210 is configurable based on the components of system 100 and the functionalities they are intended to perform.

For example, controller 210 can be operatively connected to a processing device in a processing platform which comprises a processor coupled to a memory. The processor may comprise a microprocessor, a microcontroller, an application-specific integrated circuit (ASIC), a field-programmable gate array (FPGA) or other type of processing circuitry, as well as portions or combinations of such circuitry elements. The memory may comprise random access memory (RAM), read-only memory (ROM) or other types of memory, in any combination.

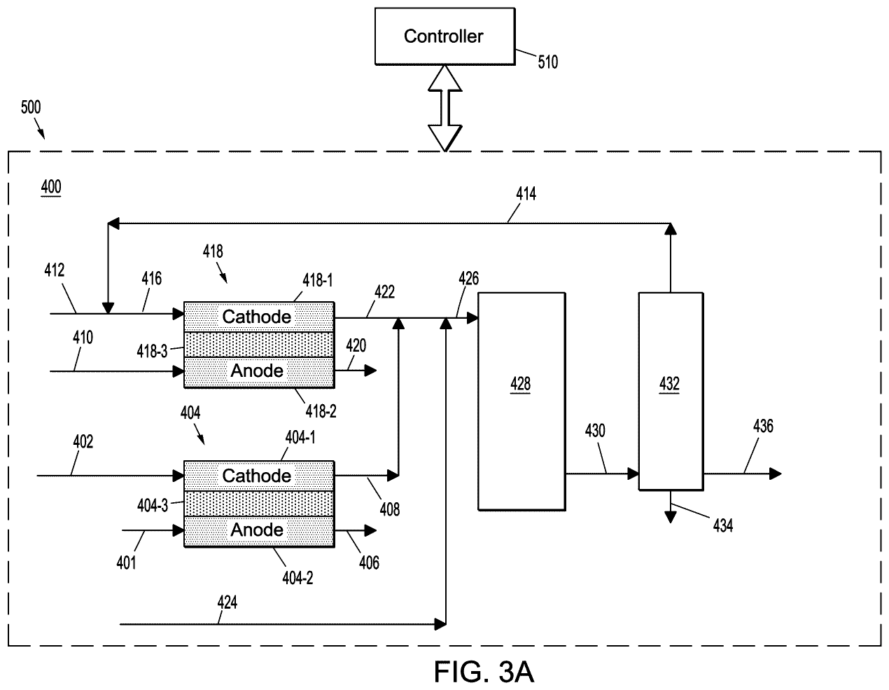

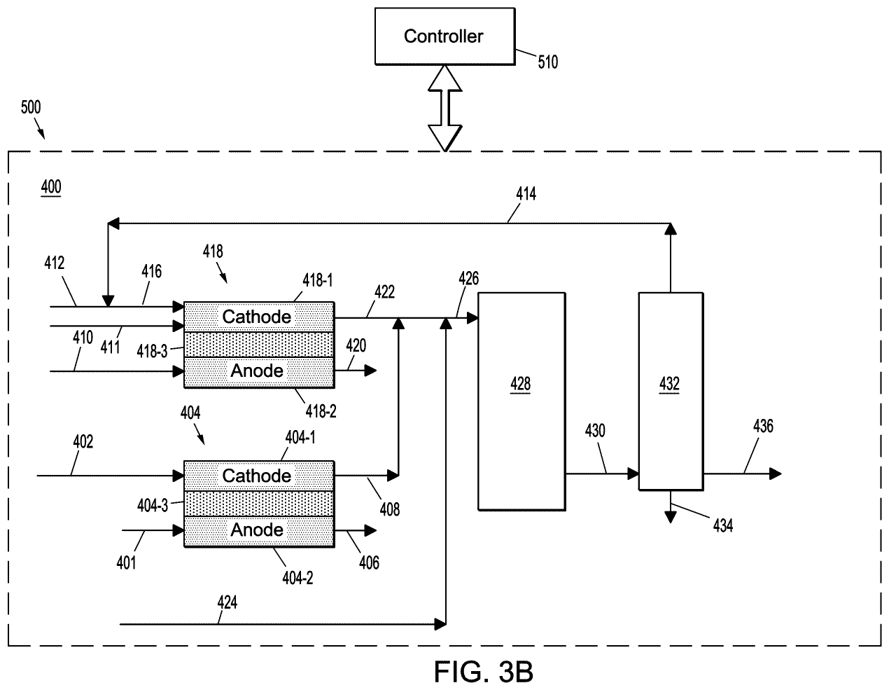

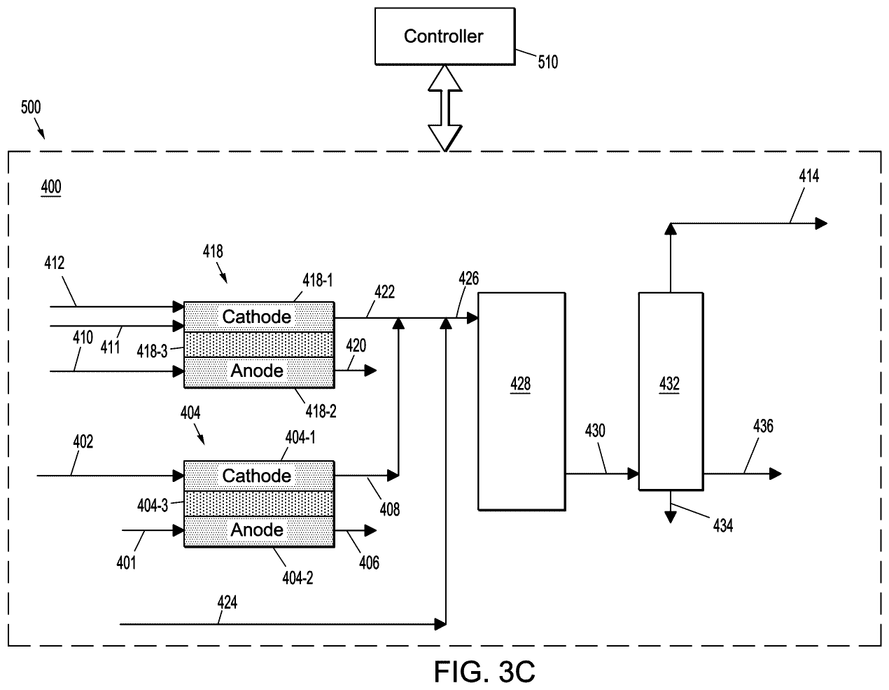

In non-limiting illustrative embodiments, a method and system for steam electrolysis of a steam feed stream in a cathode to produce hydrogen (H₂) in a first high-temperature solid-oxide electrolyzer, and steam electrolysis of a steam feed and a reforming reaction of one or more of a recycled tail gas stream from a reactor unit and a methane-rich feed stream in the cathode to produce syngas in a second high-temperature solid-oxide electrolyzer is described with reference to FIGS. 3A-3C.

Referring now to FIGS. 3A-3C, an anode purge stream 401 and a steam feed stream 402 are sent to a system 400. Anode purge stream 401 includes one or more of air, carbon dioxide or an inert gas such as N₂. Steam feed stream 402 can be obtained from any means known in the art. In some embodiments, anode purge stream 401 such as air may be pressurized to produce a pressurized anode purge stream as described above for anode purge stream 101.

System 400 includes a first electrolyzer 404 comprising a cathode 404-1, an anode 404-2 and an electrolyte 404-3 inserted between cathode 404-1 and anode 404-2. First electrolyzer 404 receives anode purge stream 401 into anode 404-2 and steam feed stream 402 into cathode 404-1. In some embodiments, steam feed stream 402 is converted to a cathode effluent 408 composed mainly of hydrogen (H₂), and an anode effluent 406 composed of at least oxygen, for example, less than 50% of oxygen each having a temperature of about 750° C. to about 850° C. For example, steam feed stream 402 is converted to cathode effluent 408 composed mainly of hydrogen (H₂) at cathode 404-1 and anode purge stream 401 serves as a purge gas to carry oxygen generated at anode 404-2 to produce anode effluent 406. Anode effluent 406 can be discarded or recycled back to first electrolyzer 404. First electrolyzer 404 can be any suitable high temperature electrolyzer as discussed above for electrolyzer 110.

System 400 further includes a second electrolyzer 418 comprising a cathode 418-1, an anode 418-2 and an electrolyte 418-3 inserted between cathode 418-1 and anode 418-2. In some embodiments, as depicted in FIG. 3A, second electrolyzer 418 receives a cathode feed stream 416 composed of a steam feed stream 412 and a recycled tail gas stream 414 containing methane and light hydrocarbons (e.g., CₓH₍₂ₓ₊₂₎ such as C₂ to C₄) from a separation unit 432 as discussed below into cathode 418-1 and an anode purge stream 410 into anode 418-2. Recycled tail gas stream 414 can reach a temperature of about 750° C. to about 850° C. after heat integration with second electrolyzer 418. For example, recycled tail gas stream 414 can be passed through one or more heat exchangers (not shown) to increase its temperature to a temperature of about 750° C. to about 850° C. for heat integration with second electrolyzer 418.

In some embodiments, as depicted in FIG. 3B, second electrolyzer 418 receives cathode feed stream 416 composed of steam feed stream 412 and recycled tail gas stream 414, and a methane-rich feed stream 411 containing methane and light hydrocarbons (e.g., CₓH₍₂ₓ₊₂₎ such as C₂ to C₄) into cathode 418-1 and anode purge stream 410 into anode 418-2. In some embodiments, methane-rich feed stream 411 can be derived from a light hydrocarbon feed stream comprising methane or natural gas or renewable natural gas such as, for example, a light hydrocarbon feed stream comprising greater than about 50%, or greater than about 80%, or greater than about 90%, or greater than about 95%, or greater than about 99% methane. As used herein, natural gas or renewable natural gas comprises methane and potentially higher alkanes, carbon dioxide, nitrogen or other gases, and/or sulfur-containing compounds such as hydrogen sulfide, and mixtures thereof.

In some embodiments, as depicted in FIG. 3C, second electrolyzer 418 receives steam feed stream 412 and methane-rich feed stream 411 into cathode 418-1 and anode purge stream 410 into anode 418-2. FIG. 3C further shows recycled tail gas stream 414 exiting separation unit 432 where it can be further processed or reused as, for example, a fuel gas.

Although steam feed stream 412 and recycled tail gas stream 414 are shown entering cathode 418-1 as cathode feed stream 416, this is merely illustrative and steam feed stream 412 and recycled tail gas stream 414 can each enter cathode 418-1 as a single stream or can be combined with methane-rich feed stream 411, when applicable, in any type of configuration. For example, steam feed stream 412 and methane-rich feed stream 411 can be combined as a single stream when entering cathode 418-1, or recycled tail gas stream 414 and methane-rich feed stream 411 can be combined as a single stream when entering cathode 418-1, or steam feed stream 412, recycled tail gas stream 414 and methane-rich feed stream 411 can be combined as a single stream when entering cathode 418-1.

Anode purge stream 410 includes one or more of air, carbon dioxide or an inert gas such as N₂. Steam feed stream 412 can be obtained from any means known in the art. In some embodiments, anode purge stream 410 such as air may be pressurized to produce a pressurized anode purge stream as described above for anode purge stream 101.

The methane and the other light hydrocarbon present in recycled tail gas stream 414 and/or methane-rich feed stream 411 react in cathode 418-1 via a reforming reaction such as steam methane reforming with steam feed stream 412 to produce a cathode effluent 422 having a temperature of about 750° C. to about 850° C. and composed of carbon monoxide (CO), hydrogen (H₂), water (H₂O) and carbon dioxide (CO₂). Anode purge stream 410 serves as a purge gas to carry oxygen generated at anode 418-2 to produce an anode effluent 420 composed of at least oxygen, for example, less than 50% of oxygen. Anode effluent 420 can be discarded or recycled back to second electrolyzer 418.

Second electrolyzer 418 can be any suitable high temperature electrolyzer as discussed above for electrolyzer 110.

In some embodiments, cathode effluent 408 (H₂-rich) and cathode effluent 422 (syngas (CO and H₂)-rich) each having a temperature of about 750° C. to about 850° C. can be sent to one or more heat exchangers (not shown) to remove heat from cathode effluent 408 and cathode effluent 422 before being combined with a carbon dioxide feed stream 424 to form a reactor feed stream 426 composed of carbon monoxide (CO), hydrogen (H₂), and minimal water (H₂O), if after a dehydration step, and carbon dioxide (CO₂) and having a temperature of about 200° C. to about 300° C. Carbon dioxide feed stream 424 enters system 400 at or around a temperature of about 20° C. However, after undergoing heat integration with the fuel synthesis effluent stream (i.e., a reactor synthesis effluent 430), the temperature of the carbon dioxide can increase to a range of about 200° C. to about 300° C.

System 400 further includes a reactor unit 428 for receiving reactor feed stream 426. In some embodiments, reactor unit 428 can be one or more of a Fischer-Tropsch reactor, methanol and/or dimethyl ether reactors and a hydrogenation reactor. In some embodiments, reactor unit 428 can be a Fischer-Tropsch reactor for converting syngas in reactor feed stream 426 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron- or cobalt-based catalyst) into hydrocarbon products, water and other byproducts. The Fischer-Tropsch reactor can be any of those discussed above. The products produced from the reaction process in reactor unit 428 can then be discharged from reactor unit 428 as reactor synthesis effluent 430 comprising Fischer-Tropsch products.

In some embodiments, system 400 may include a hydrocracker unit and/or fractionation unit to upgrade the Fischer-Tropsch liquids as discussed above for system 100.

In some embodiments, reactor unit 428 can be a methanol and/or dimethyl ether reactor, rather than a Fischer-Tropsch reactor for processing reactor feed stream 426. The products produced from the reaction process in reactor unit 428 can then be discharged from reactor unit 428 as reactor synthesis effluent 430 comprising one or more of methanol and/or dimethyl ether.

In some embodiments, reactor unit 428 can be a hydrogenation reactor for direct hydrogenation of reactor feed stream 426 to produce liquid hydrocarbons as discussed above.

Accordingly, for purposes of this illustrative embodiment, reactor synthesis effluent 430 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 426 is contemplated herein for use as reactor synthesis effluent 430.

The Fischer-Tropsch products, methanol/dimethyl ether products and/or direct hydrogenation products produced from the reaction process in reactor synthesis effluent 430 can then be discharged from reactor unit 428 and sent to a separation unit 432. Separation unit 432 can be any of those discussed above for separation unit 122. For example, the Fischer-Tropsch products, methanol/dimethyl ether products and/or direct hydrogenation products can be separated and collected following conventional fractional distillation to produce a product effluent 436 for sending further downstream for processing, while a portion of any unreacted syngas can be recycled to reactor unit 428 for further conversion (not shown), and any methane and light hydrocarbons such as C₁-C₄ light ends produced from the synthesis reactions can be recycled from separation unit 432 as recycled tail gas stream 414 back to cathode 418-1 of second electrolyzer 418 (see FIGS. 3A and 3B) or sent for further processing or reused as, for example, a fuel gas (see FIG. 3C) as discussed above. A liquid stream 434 including at least hydrocarbons and water can be sent for further downstream processing as known in the art. For example, product effluent 436 can be sent for further downstream processing for creating high value liquid fuels, such as gasoline, diesel, and jet, or base oils.

In one or more illustrative embodiments, a system processing environment 500 comprises each of the components of system 400 described herein, as well as a controller 510 operatively coupled to system processing environment 500. Controller 510 is configured to control operations of one or more of the components of system 400 discussed above. In one illustrative embodiment, controller 510 is configured to actuate one or more of the functionalities of system 400 described herein. For example, controller 510 can be similar to controller 210 described above and function in a similar manner.

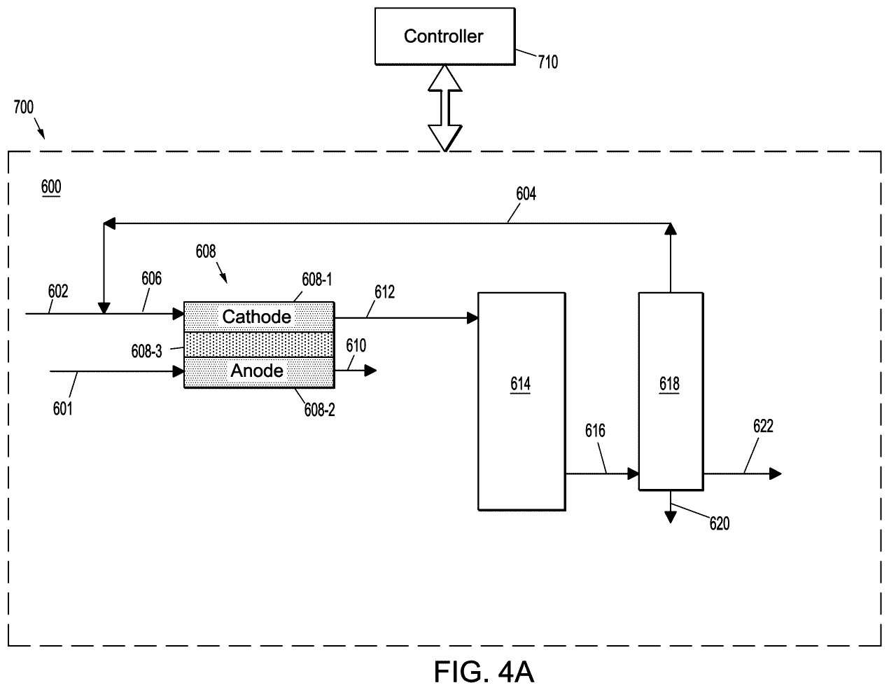

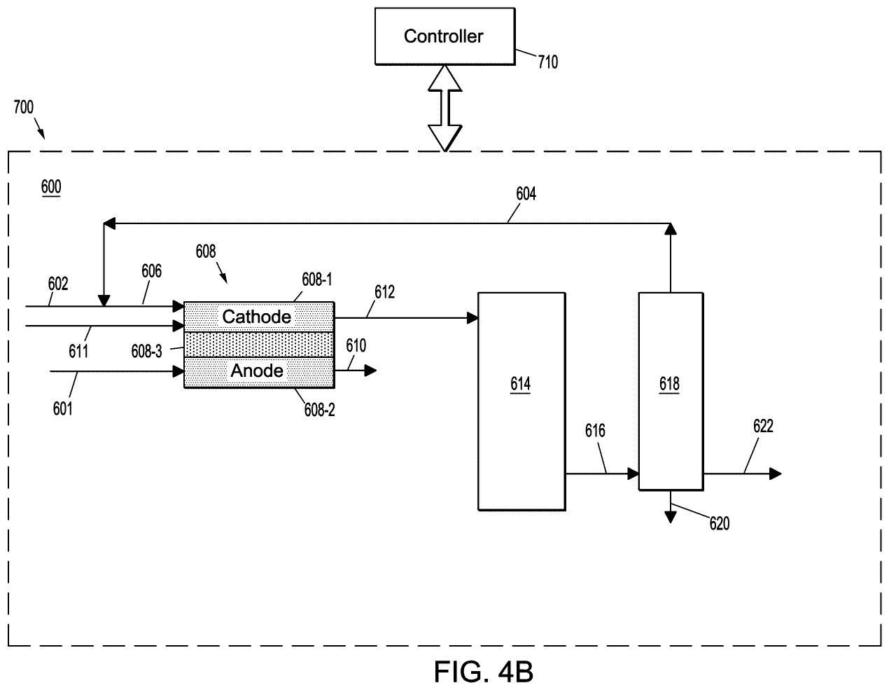

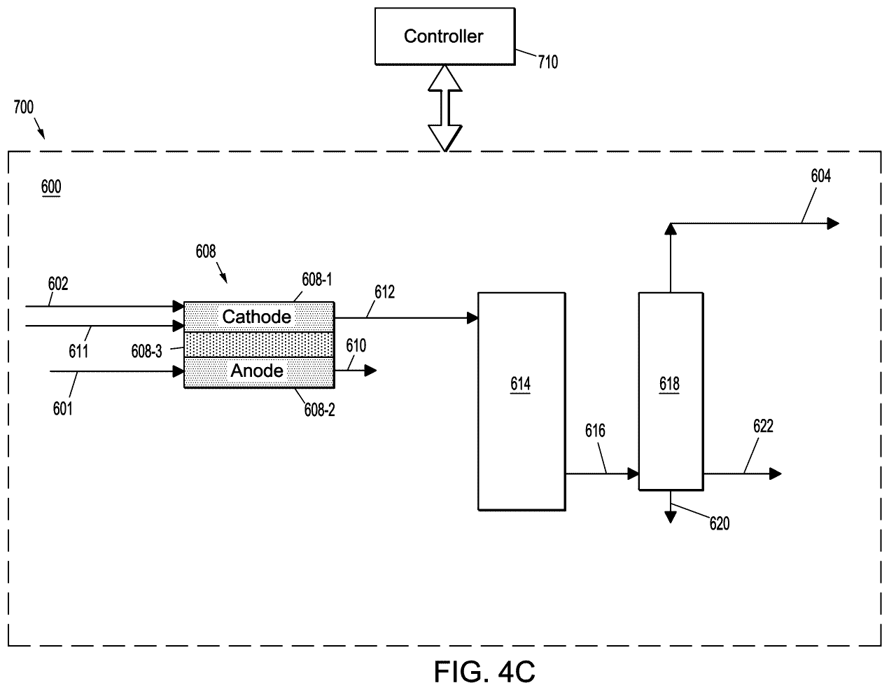

In non-limiting illustrative embodiments, a method and system for co-electrolysis of a steam and carbon dioxide feed stream to produce a mixture of carbon monoxide (CO) and hydrogen (H₂) (i.e., syngas), and a reforming reaction of one or more of a recycled tail gas stream and a methane-rich feed stream in the cathode to produce syngas in a high-temperature solid-oxide electrolyzer is described with reference to FIGS. 4A-4C.

Referring now to FIGS. 4A-4C, system 600 includes an electrolyzer 608 comprising a cathode 608-1, an anode 608-2 and an electrolyte 608-3 inserted between cathode 608-1 and anode 608-2. In some embodiments, as depicted in FIG. 4A, electrolyzer 608 receives a cathode feed stream 606 composed of a steam and carbon dioxide feed stream 602 and a recycled tail gas stream 604 containing methane and light hydrocarbons (e.g., CₓH₍₂ₓ₊₂₎ such as C₂ to C₄) from a separation unit 618 as discussed below into cathode 608-1 and an anode purge stream 601 into anode 608-2. Recycled tail gas stream 604 can reach a temperature of about 750° C. to about 850° C. after heat integration with electrolyzer 608. For example, recycled tail gas stream 604 can be passed through one or more heat exchangers (not shown) to increase its temperature to a temperature of about 750° C. to about 850° C. for heat integration with electrolyzer 608.

In some embodiments, as depicted in FIG. 4B, electrolyzer 608 receives cathode feed stream 606 composed of steam and carbon dioxide feed stream 602 and recycled tail gas stream 604, and a methane-rich feed stream 611 containing methane and light hydrocarbons (e.g., CₓH₍₂ₓ₊₂₎ such as C₂ to C₄) into cathode 608-1 and anode purge stream 601 into anode 608-2. In some embodiments, methane-rich feed stream 611 can be derived from a light hydrocarbon feed stream comprising methane or natural gas or renewable natural gas such as, for example, a light hydrocarbon feed stream comprising greater than about 50%, or greater than about 80%, or greater than about 90%, or greater than about 95%, or greater than about 99% methane. As used herein, natural gas or renewable natural gas comprises methane and potentially higher alkanes, carbon dioxide, nitrogen or other gases, and/or sulfur-containing compounds such as hydrogen sulfide, and mixtures thereof.

In some embodiments, as depicted in FIG. 4C, electrolyzer 608 receives steam and carbon dioxide feed stream 602 and methane-rich feed stream 611 into cathode 608-1 and anode purge stream 601 into anode 608-2 and recycled tail gas stream 604 exits separation unit 618. FIG. 4C further shows recycled tail gas stream 604 exiting separation unit 618 where it can be further processed or reused as, for example, a fuel gas.

Although steam and carbon dioxide feed stream 602 and recycled tail gas stream 604 are shown entering cathode 608-1 as cathode feed stream 606, this is merely illustrative and steam and carbon dioxide feed stream 602 and recycled tail gas stream 604 can each enter cathode 608-1 as a single stream or can be combined with methane-rich feed stream 611, when applicable, in any type of configuration. For example, steam and carbon dioxide feed stream 602 and methane-rich feed stream 611 can be combined as a single stream when entering cathode 608-1, or recycled tail gas stream 604 and methane-rich feed stream 611 can be combined as a single stream when entering cathode 608-1, or steam and carbon dioxide feed stream 602, recycled tail gas stream 604 and methane-rich feed stream 611 can be combined as a single stream when entering cathode 608-1.

Anode purge stream 601 includes one or more of air, carbon dioxide or an inert gas such as N₂. Steam and carbon dioxide feed stream 602 can be obtained from any means known in the art. In some embodiments, anode purge stream 601 such as air may be pressurized to produce a pressurized anode purge stream as described above for anode purge stream 101.

Electrolyzer 608 can operate in a co-electrolysis mode in which steam and carbon dioxide feed stream 602 participates in a reaction to produce a cathode effluent 612 including syngas composed of carbon monoxide (CO) and hydrogen (H₂) and anode purge stream 601 into anode 608-2 to produce an anode effluent (oxygen enriched stream) 610 from anode 608-2. Anode purge stream 601 serves as a purge gas to carry oxygen generated at anode 608-2. Anode effluent 610 can be discarded or recycled back to electrolyzer 608.

In an illustrative embodiment, electrolyzer 608 can be any suitable high temperature electrolyzer comprising cathode 608-1, anode 608-2 and electrolyte 608-3 inserted between anode 608-2 and cathode 608-1. In a non-limiting illustrative embodiment, electrolyzer 608 is a high temperature solid oxide electrolyzer (also referred to as SOEC) comprising:
a first porous conductive electrode, or “cathode,” to be supplied with steam and carbon dioxide for the production of dihydrogen and carbon monoxide;
a second porous conductive electrode, or “anode,” via which the dioxygen (O₂) produced by the electrolysis of the water injected onto the cathode escapes; and
a solid oxide membrane (dense electrolyte) sandwiched between the cathode and the anode, the membrane being anionically conductive at high temperatures, usually temperatures above about 700° C. and up to about 950° C.

Electrolyzer 608 may receive input energy (i.e., electricity) from an intermittent source such as solar power (including photovoltaic and reflective), wind power, tidal power, wave power, batteries, and other intermittent energy sources known in the art and combinations thereof. Alternatively, or in addition, electrolyzer 608 may receive input energy from a non-intermittent source, such as an electricity grid (e.g., a regional electricity grid, a municipal electricity grid, or a microgrid), natural gas, coal, nuclear, and other non-intermittent sources known in the art and combinations thereof. Electrolyzer 608 may therefore be electricity connectable to an intermittent energy input, a non-intermittent source, or a combination thereof. In particular embodiments, electrolyzer 608 may receive input energy from the photovoltaic panel.

In some embodiments, electrolyzer 608 may be operational receiving electricity from a photovoltaic panel. At night, electrolyzer 608 may be operated in “hot stand-by” mode to conserve electricity, or electrolyzer 608 may be electricity connected to another power source to continue operating at night. In particular, electrolyzer 608 may be connected to a power grid such as a regional power grid, a municipal power grid, or a micro grid, and electrolyzer 608 may run when the price of electricity is low.

System 600 may further comprise an energy storage mechanism or a plurality of energy storage mechanisms. The energy storage mechanism may comprise any mechanism or apparatus operable to store energy such as electricity, thermal energy, etc. For example, the energy storage mechanism may include batteries (e.g., lead-acid batteries, lithium-ion batteries, lithium iron batteries, etc.), water, flywheels, compressed air, pumped hydroelectric, or other energy storage mechanisms known in the art and combinations thereof.

In illustrative embodiments, a solid oxide electrolyzer can include a stack of elementary solid-oxide (co-)electrolysis cells each comprising an anode, a cathode, and an electrolyte inserted between the anode and the cathode, the cells being electrically connected in series, the stack comprising two electrical terminals for the supply of current to the cells and defining flow chambers for, with respect to the first chambers, the flow of steam, hydrogen and carbon dioxide, and carbon monoxide over the cathodes and flow chambers for, with respect to the second chambers, the flow of air or nitrogen or oxygen or of a mixture of gases containing oxygen, and carbon dioxide over the anodes.

In illustrative embodiments, a solid oxide electrolyzer generally relies on an electron source (external source of electricity). The heat and electricity to operate the solid oxide electrolyzer may be produced from renewable sources, such as solar, wind, geothermal, or hydropower. Heat may be added to the solid oxide electrolyzer to maintain a desired operating temperature of the solid oxide electrolyzer including the electrochemical reduction. In an illustrative embodiment, heat may be added to the solid oxide electrolyzer by, for example, resistive heating (e.g., at the solid oxide electrolyzer electrodes), a steam jacket, solar heating systems, etc.

A suitable operating temperature and pressure of the solid oxide electrolyzer and the material of the solid oxide electrolyzer electrodes for electrolyzer 608 can be any of those discussed above for electrolyzer 110.

The methane and the other light hydrocarbon present in recycled tail gas stream 604 and/or methane-rich feed stream 611 react in cathode 608-1 via a reforming reaction such as, for example, steam methane reforming, dry reforming, etc., together with steam and carbon dioxide feed stream 602 utilizing a co-electrolysis mode to produce cathode effluent 612 further composed of at least syngas composed of mostly carbon monoxide (CO) and hydrogen (H₂) along with water (H₂O) and carbon dioxide (CO₂) having a temperature of about 750° C. to about 850° C.

An advantage of the illustrative embodiments described herein is that by using co-electrolysis the composition of the syngas feed to a reactor unit 614 as discussed below can be optimized for the target synthesis reaction to achieve better performance such as higher conversion and product selectivity. For example, since syngas is produced by co-electrolysis of steam and carbon dioxide feed stream 602 as well as a reforming reaction of one or more of recycled tail gas stream 604 and methane-rich feed stream 611 including light hydrocarbons, a Fisher-Tropsch reactor or a methanol reactor can be further used to convert syngas into hydrocarbons or methanol/DME.

System 600 further includes a reactor unit 614 for receiving cathode effluent 612. In some embodiments, reactor unit 614 can be one or more of a Fischer-Tropsch reactor, methanol and/or dimethyl ether reactors and a hydrogenation reactor. In some embodiments, reactor unit 614 can be a Fischer-Tropsch reactor for converting syngas in cathode effluent 612 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron- or cobalt-based catalyst) into hydrocarbon products, water and other byproducts. The Fischer-Tropsch reactor can be any of those discussed above. The products produced from the reaction process in reactor unit 614 can then be discharged from reactor unit 614 as a reactor synthesis effluent 616 comprising Fischer-Tropsch products.

In some embodiments, reactor unit 614 can be a methanol and/or dimethyl ether reactor, rather than a Fischer-Tropsch reactor for processing cathode effluent 612. The products produced from the reaction process in reactor unit 614 can then be discharged from reactor unit 614 as reactor synthesis effluent 616 comprising one or more of methanol and/or dimethyl ether.

In some embodiments, reactor unit 614 can be a hydrogenation reactor for the direct hydrogenation of cathode effluent 612 to produce liquid hydrocarbons as discussed above.

Accordingly, for purposes of this illustrative embodiment, reactor synthesis effluent 616 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of cathode effluent 612 is contemplated herein for use as reactor synthesis effluent 616.

The Fischer-Tropsch products, methanol/dimethyl ether products and/or direct hydrogenation products produced from the reaction process in reactor synthesis effluent 616 can then be discharged from reactor unit 614 and sent to a separation unit 618. Separation unit 618 can be any of those discussed above for separation unit 122. For example, the Fischer-Tropsch products, methanol/dimethyl ether products and/or direct hydrogenation products can be separated and collected following conventional fractional distillation to produce a product effluent 622 for sending further downstream for processing, while a portion of any unreacted syngas can be recycled to reactor unit 614 for further conversion (not shown), and any methane and light hydrocarbons such as C₁ to C₄ light ends produced from the synthesis reactions can be recycled from separation unit 618 as recycled tail gas stream 604 back to cathode 608-1 of electrolyzer 608 (see FIGS. 4A and 4B) or sent for further processing or reused as, for example, a fuel gas (see FIG. 4C) as discussed above. A liquid stream 620 including at least hydrocarbons and water is sent for further downstream processing as known in the art. For example, product effluent 622 can be sent for further downstream processing for creating high value liquid fuels, such as gasoline, diesel, and jet, or base oils.

In some embodiments, system 600 may include a hydrocracker unit and/or fractionation unit to upgrade product effluent 622 such as the Fischer-Tropsch liquids as discussed above for system 100.

In one or more illustrative embodiments, a system processing environment 700 comprises each of the components of system 600 described herein, as well as a controller 710 operatively coupled to system processing environment 700. Controller 710 is configured to control operations of one or more of the components of system 600 discussed above. In one illustrative embodiment, controller 710 is configured to actuate one or more of the functionalities of system 600 described herein. For example, controller 710 can be similar to controller 210 described above and function in a similar manner.

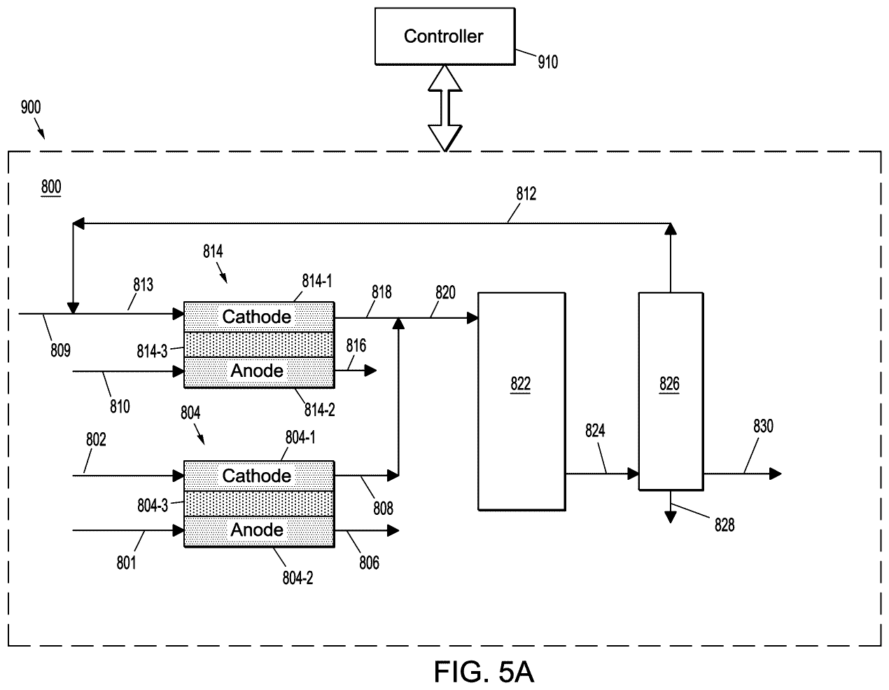

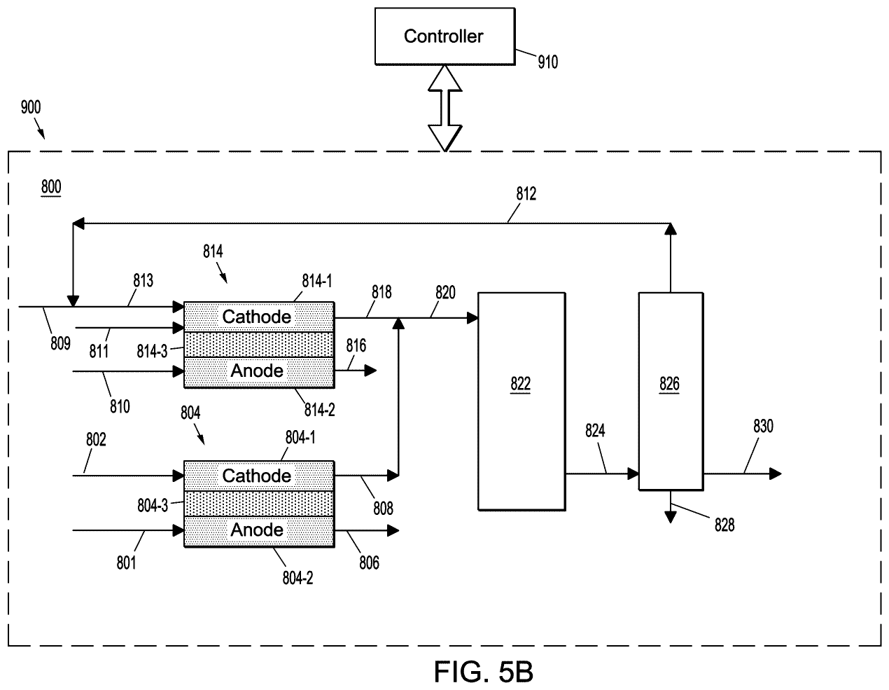

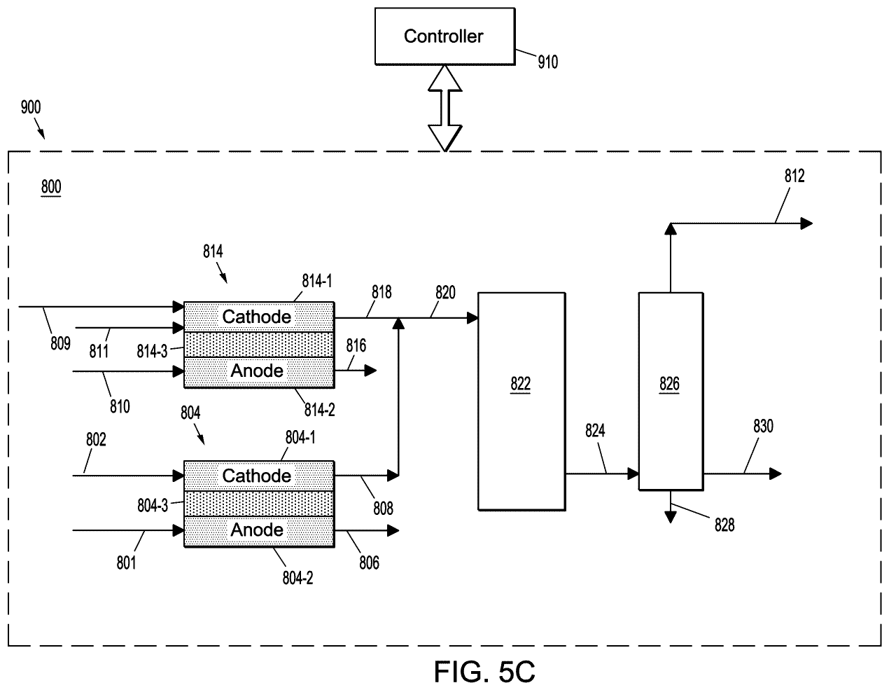

In non-limiting illustrative embodiments, a method and system for co-electrolysis of a steam and carbon dioxide feed stream to produce syngas in a cathode in a first high-temperature solid-oxide electrolyzer and for co-electrolysis of a steam and carbon dioxide feed stream to produce syngas, and a reforming reaction of a recycled tail gas stream and/or a methane-rich feed stream in the cathode to produce syngas in a second high-temperature solid-oxide electrolyzer is described with reference to FIGS. 5A-5C.

Referring now to FIGS. 5A-5C, a system 800 includes a first electrolyzer 804 comprising a cathode 804-1, an anode 804-2 and an electrolyte 804-3 inserted between cathode 804-1 and anode 804-2. First electrolyzer 804 receives a steam and carbon dioxide feed stream 802 into cathode 804-1 of first electrolyzer 804 and an anode purge stream 801 in anode 804-2 of first electrolyzer 804. Anode purge stream 801 includes one or more of air, carbon dioxide or an inert gas such as N₂. In some embodiments, anode purge stream 801 such as air may be pressurized to produce a pressurized anode purge stream as described above for anode purge stream 101.

First electrolyzer 804 can operate in a co-electrolysis mode in which steam and carbon dioxide feed stream 802 participates in a reaction to produce a cathode effluent 808 including syngas composed of carbon monoxide (CO) and hydrogen (H₂) and anode purge stream 801 into anode 804-2 to produce an anode effluent (oxygen enriched stream) 806 from anode 804-2. Anode purge stream 801 serves as a purge gas to carry oxygen generated at anode 804-2. Anode effluent 806 can be discarded or recycled back to first electrolyzer 804.

First electrolyzer 804 can be any suitable high temperature electrolyzer as discussed above for electrolyzer 608.

System 800 further includes a second electrolyzer 814 comprising a cathode 814-1, an anode 814-2 and an electrolyte 814-3 inserted between cathode 814-1 and anode 814-2. In some embodiments, as depicted in FIG. 5A, second electrolyzer 814 receives a cathode feed stream 813 composed of a steam and carbon dioxide feed stream 809 and a recycled tail gas stream 812 containing methane and light hydrocarbons (e.g., CₓH₍₂ₓ₊₂₎ such as C₂ to C₄) from a separation unit 826 as discussed below into cathode 814-1 and an anode purge stream 810 in anode 814-2. Recycled tail gas stream 812 can reach a temperature of about 750° C. to about 850° C. after heat integration with second electrolyzer 814. For example, recycled tail gas stream 812 can be passed through one or more heat exchangers (not shown) to increase its temperature to a temperature of about 750° C. to about 850° C. for heat integration with second electrolyzer 814.

In some embodiments, as depicted in FIG. 5B, second electrolyzer 814 receives cathode feed stream 813 composed of steam and carbon dioxide feed stream 809, recycled tail gas stream 812, and a methane-rich feed stream 811 containing methane and light hydrocarbons (e.g., CₓH₍₂ₓ₊₂₎ such as C₂ to C₄) into cathode 814-1 and anode purge stream 810 into anode 814-2. In some embodiments, methane-rich feed stream 811 can be derived from a light hydrocarbon feed stream comprising methane or natural gas or renewable natural gas such as, for example, a light hydrocarbon feed stream comprising greater than about 50%, or greater than about 80%, or greater than about 90%, or greater than about 95%, or greater than about 99% methane. As used herein, natural gas or renewable natural gas comprises methane and potentially higher alkanes, carbon dioxide, nitrogen or other gases, and/or sulfur-containing compounds such as hydrogen sulfide, and mixtures thereof.

In some embodiments, as depicted in FIG. 5C, second electrolyzer 814 receives steam and carbon dioxide feed stream 809 and methane-rich feed stream 811 into cathode 814-1 and anode purge stream 810 into anode 814-2. FIG. 5C further shows recycled tail gas stream 812 exiting separation unit 826 where it can be further processed or reused as, for example, a fuel gas.

Although steam and carbon dioxide feed stream 809 and recycled tail gas stream 812 are shown entering cathode 814-1 as cathode feed stream 813, this is merely illustrative and steam and carbon dioxide feed stream 809 and recycled tail gas stream 812 can each enter cathode 814-1 as a single stream or can be combined with methane-rich feed stream 811, when applicable, in any type of configuration. For example, steam and carbon dioxide feed stream 809 and methane-rich feed stream 811 can be combined as a single stream when entering cathode 814-1, or recycled tail gas stream 812 and methane-rich feed stream 811 can be combined as a single stream when entering cathode 814-1, or steam and carbon dioxide feed stream 809, recycled tail gas stream 812 and methane-rich feed stream 811 can be combined as a single stream when entering cathode 814-1.

Anode purge stream 810 includes one or more of air, carbon dioxide or an inert gas such as N₂. Steam and carbon dioxide feed stream 809 can be obtained from any means known in the art. In some embodiments, anode purge stream 810 such as air may be pressurized to produce a pressurized anode purge stream as described above for anode purge stream 101.

Second electrolyzer 814 can operate in a co-electrolysis mode in which steam and carbon dioxide feed stream 809 participates in a reaction to produce a cathode effluent 818 including syngas composed of carbon monoxide (CO) and hydrogen (H₂) and an anode purge stream 810 into anode 814-2 to produce an anode effluent (oxygen enriched stream) 816 from anode 814-2. Anode purge stream 810 serves as a purge gas to carry oxygen generated at anode 814-2. Anode effluent 816 can be discarded or recycled back to second electrolyzer 814.

The methane and the other light hydrocarbon present in recycled tail gas stream 812 and/or methane-rich feed stream 811 react in cathode 814-1 via a reforming reaction such as, for example, steam methane reforming, dry reforming, etc., with steam in the steam and carbon dioxide feed stream 809 to produce cathode effluent 818 further composed of at least syngas composed of mostly carbon monoxide (CO) and hydrogen (H₂) along with water (H₂O) and carbon dioxide (CO₂) having a temperature of about 750° C. to about 850° C.

Second electrolyzer 814 can be any suitable high temperature electrolyzer as discussed above for electrolyzer 608.

Cathode effluent 808 (syngas (CO and H₂)-rich) and cathode effluent 818 (syngas (CO and H₂)-rich) each having a temperature of about 750° C. to about 850° C. can be sent to one or more heat exchangers (not shown) to remove heat from cathode effluent 808 and cathode effluent 818 before being combined to form a reactor feed stream 820 having a temperature of about 200° C. to about 300° C.

System 800 further includes a reactor unit 822 for receiving reactor feed stream 820. In some embodiments, reactor unit 822 can be one or more of a Fischer-Tropsch reactor, methanol and/or dimethyl ether reactors and a hydrogenation reactor. In some embodiments, reactor unit 822 can be a Fischer-Tropsch reactor for converting syngas in reactor feed stream 820 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron- or cobalt-based catalyst) into hydrocarbon products, water and other byproducts. The Fischer-Tropsch reactor can be any of those discussed above. The products produced from the reaction process in reactor unit 822 can then be discharged from reactor unit 822 as a reactor synthesis effluent 824 comprising Fischer-Tropsch products.

In some embodiments, reactor unit 822 can be a methanol and/or dimethyl ether reactor, rather than a Fischer-Tropsch reactor for processing reactor feed stream 820. The products produced from the reaction process in reactor unit 822 can then be discharged from reactor unit 822 as reactor synthesis effluent 824 comprising one or more of methanol and/or dimethyl ether.

In some embodiments, reactor unit 822 can be a hydrogenation reactor for the direct hydrogenation of reactor feed stream 820 to produce liquid hydrocarbons as discussed above.

Accordingly, for purposes of this illustrative embodiment, reactor synthesis effluent 824 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 820 is contemplated herein for use as reactor synthesis effluent 824.

The Fischer-Tropsch products, methanol/dimethyl ether products and/or direct hydrogenation products produced from the reaction process in reactor synthesis effluent 824 can then be discharged from reactor unit 822 and sent to separation unit 826. Separation unit 826 can be any of those discussed above for separation unit 122. For example, the Fischer-Tropsch products, methanol/dimethyl ether products and/or direct hydrogenation products can be separated and collected following conventional fractional distillation to produce a product effluent 830 for sending further downstream for processing, while a portion of any unreacted syngas can be recycled to reactor unit 822 for further conversion not shown), and any methane and light hydrocarbons such as C₁-C₄ light ends produced from the synthesis reactions can be recycled from separation unit 826 as recycled tail gas stream 812 back to cathode 814-1 of second electrolyzer 814 (see FIGS. 5A and 5B) or sent for further processing or reused as, for example, a fuel gas (see FIG. 5C) as discussed above. A liquid stream 828 including at least hydrocarbons and water is sent for further downstream processing as known in the art. For example, product effluent 830 can be sent for further downstream processing for creating high value liquid fuels, such as gasoline, diesel, and jet, or base oils.

In some embodiments, system 800 may include a hydrocracker unit and/or fractionation unit to upgrade the Fischer-Tropsch liquids as discussed above for system 100.

In one or more illustrative embodiments, a system processing environment 900 comprises each of the components of system 800 described herein, as well as a controller 910 operatively coupled to system processing environment 900. Controller 910 is configured to control operations of one or more of the components of system 800 discussed above. In one illustrative embodiment, controller 910 is configured to actuate one or more of the functionalities of system 800 described herein. For example, controller 910 can be similar to controller 210 described above and function in a similar manner.

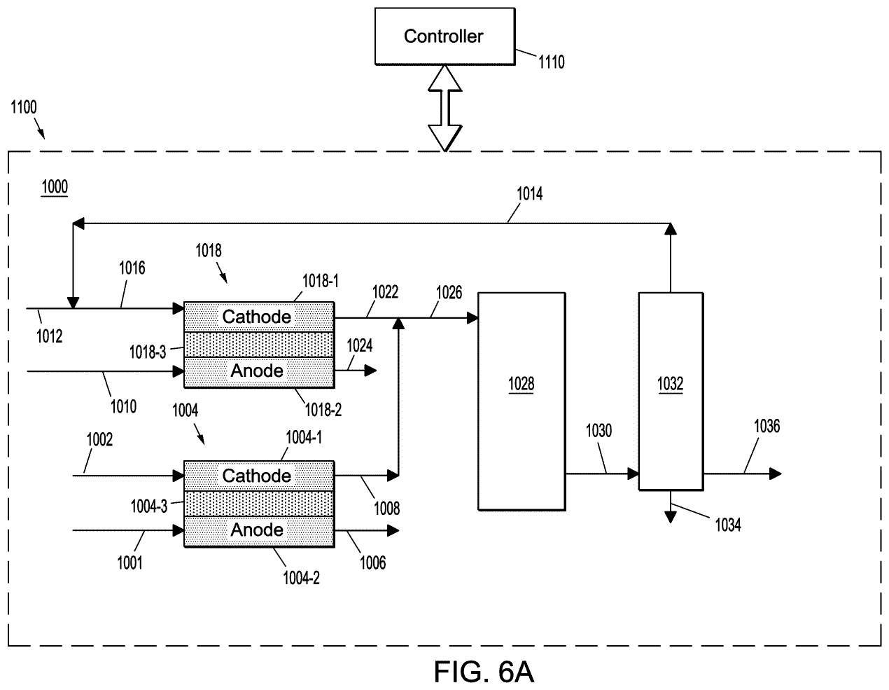

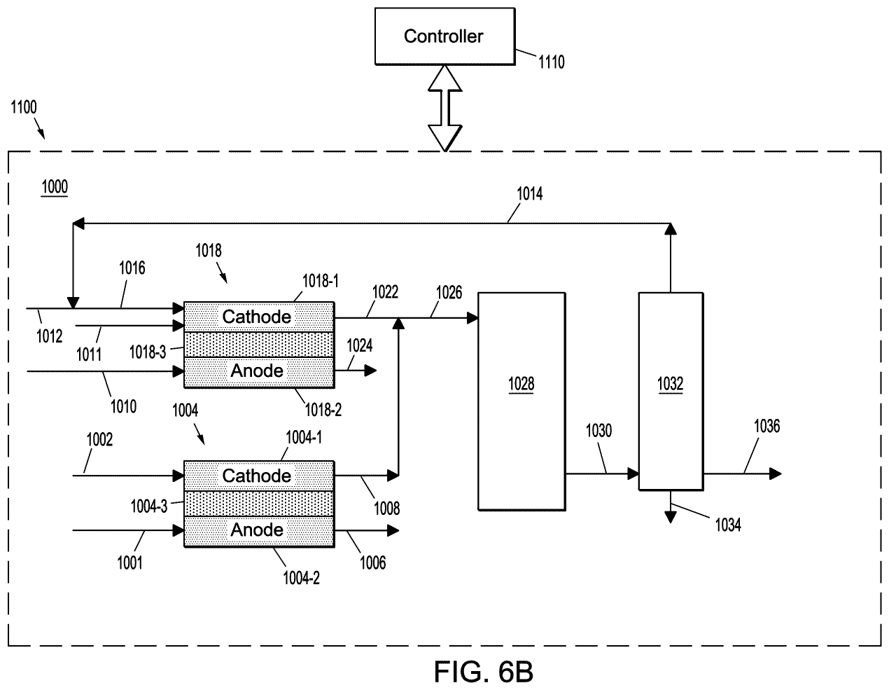

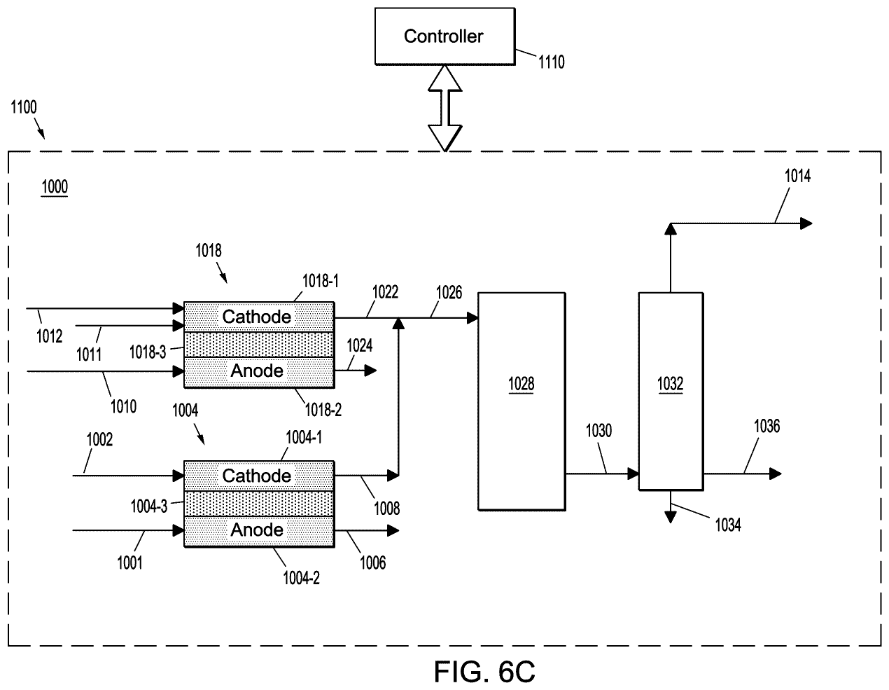

In non-limiting illustrative embodiments, a method and system for co-electrolysis of a steam and carbon dioxide feed stream to produce syngas in a cathode in a first high-temperature solid-oxide electrolyzer, and steam electrolysis of a steam feed and a reforming reaction of one or more of a recycled tail gas stream from a reactor unit and a methane-rich feed stream in the cathode to produce syngas in a second high-temperature solid-oxide electrolyzer is described with reference to FIGS. 6A-6C.

Referring now to FIGS. 6A-6C, a system 1000 includes a first electrolyzer 1004 comprising a cathode 1004-1, an anode 1004-2 and an electrolyte 1004-3 inserted between cathode 1004-1 and anode 1004-2. First electrolyzer 1004 receives a steam and carbon dioxide feed stream 1002 into cathode 1004-1 of first electrolyzer 1004 and an anode purge stream 1001 in anode 1004-2 of first electrolyzer 1004. Anode purge stream 1001 includes one or more of air, carbon dioxide or an inert gas such as N₂. In some embodiments, anode purge stream 1001 such as air may be pressurized to produce a pressurized anode purge stream as described above for anode purge stream 101.

First electrolyzer 1004 can operate in a co-electrolysis mode in which steam and carbon dioxide feed stream 1002 participates in a reaction to produce a cathode effluent 1008 including syngas composed of carbon monoxide (CO) and hydrogen (H₂) and anode purge stream 1001 into anode 1004-2 to produce an anode effluent (oxygen enriched stream) 1006 from anode 1004-2. Anode purge stream 1001 serves as a purge gas to carry oxygen generated at anode 1004-2. Anode effluent 1006 can be discarded or recycled back to first electrolyzer 1004.

First electrolyzer 1004 can be any suitable high temperature electrolyzer as discussed above for electrolyzer 608.

System 1000 further includes a second electrolyzer 1018 comprising a cathode 1018-1, an anode 1018-2 and an electrolyte 1018-3 inserted between cathode 1018-1 and anode 1018-2. In some embodiments, as depicted in FIG. 6A, second electrolyzer 1018 receives a cathode feed stream 1016 composed of a steam feed stream 1012 and a recycled tail gas stream 1014 containing methane and light hydrocarbons (e.g., CₓH₍₂ₓ₊₂₎ such as C₂ to C₄) from a separation unit 1032 as discussed below into cathode 1018-1 and an anode purge stream 1010 in anode 1018-2. Recycled tail gas stream 1014 can reach a temperature of about 750° C. to about 850° C. after heat integration with second electrolyzer 1018. For example, recycled tail gas stream 1014 can be passed through one or more heat exchangers (not shown) to increase its temperature to a temperature of about 750° C. to about 850° C. for heat integration with second electrolyzer 1018.

In some embodiments, as depicted in FIG. 6B, second electrolyzer 1018 receives cathode feed stream 1016 composed of steam feed stream 1012, recycled tail gas stream 1014, and a methane-rich feed stream 1011 containing methane and light hydrocarbons (e.g., CₓH₍₂ₓ₊₂₎ such as C₂ to C₄) into cathode 1018-1 and anode purge stream 1010 into anode 1018-2. In some embodiments, methane-rich feed stream 1011 can be derived from a light hydrocarbon feed stream comprising methane or natural gas or renewable natural gas such as, for example, a light hydrocarbon feed stream comprising greater than about 50%, or greater than about 80%, or greater than about 90%, or greater than about 95%, or greater than about 99% methane. As used herein, natural gas or renewable natural gas comprises methane and potentially higher alkanes, carbon dioxide, nitrogen or other gases, and/or sulfur-containing compounds such as hydrogen sulfide, and mixtures thereof.

In some embodiments, as depicted in FIG. 6C, second electrolyzer 1018 receives steam feed stream 1012 and methane-rich feed stream 1011 into cathode 1018-1 and anode purge stream 1010 into anode 1018-2. FIG. 6C further shows recycled tail gas stream 1014 exiting separation unit 1032 where it can be further processed or reused as, for example, a fuel gas.

Although steam feed stream 1012 and recycled tail gas stream 1014 are shown entering cathode 1018-1 as cathode feed stream 1016, this is merely illustrative and steam feed stream 1012 and recycled tail gas stream 1014 can each enter cathode 1018-1 as a single stream or can be combined with methane-rich feed stream 1011, when applicable, in any type of configuration. For example, steam feed stream 1012 and methane-rich feed stream 1011 can be combined as a single stream when entering cathode 1018-1, or recycled tail gas stream 1014 and methane-rich feed stream 1011 can be combined as a single stream when entering cathode 1018-1, or steam feed stream 1012, recycled tail gas stream 1014 and methane-rich feed stream 1011 can be combined as a single stream when entering cathode 1018-1.

Anode purge stream 1010 includes one or more of air, carbon dioxide or an inert gas such as N₂. In some embodiments, anode purge stream 1010 such as air may be pressurized to produce a pressurized anode purge stream as described above for anode purge stream 101.

The methane and the other light hydrocarbon present in recycled tail gas stream 1014 and/or methane-rich feed stream 1011 react in cathode 1018-1 via a reforming reaction such as steam methane reforming with steam feed stream 1012 to produce a cathode effluent 1022 having a temperature of about 750° C. to about 850° C. and composed of carbon monoxide (CO), hydrogen (H₂), water (H₂O) and carbon dioxide (CO₂). Anode purge stream 1010 serves as a purge gas to carry oxygen generated at anode 1018-2 to produce an anode effluent 1024 composed of at least oxygen, for example, less than 50% of oxygen. Anode effluent 1024 can be discarded or recycled back to second electrolyzer 1018.

Second electrolyzer 1018 can be any suitable high temperature electrolyzer as discussed above for electrolyzer 110.

Cathode effluent 1008 (syngas (CO and H₂)-rich) and cathode effluent 1022 (syngas (CO and H₂)-rich) each having a temperature of about 750° C. to about 850° C. can be sent to one or more heat exchangers (not shown) to remove heat from cathode effluent 1008 and cathode effluent 1022 before combined to form a reactor feed stream 1026 composed of carbon monoxide (CO), hydrogen (H₂), and minimal water (H₂O) if after a dehydration step and carbon dioxide (CO₂) and having a temperature of about 200° C. to about 300° C.

System 1000 further includes a reactor unit 1028 for receiving reactor feed stream 1026. In some embodiments, reactor unit 1028 can be one or more of a Fischer-Tropsch reactor, methanol and/or dimethyl ether reactors and a hydrogenation reactor. In some embodiments, reactor unit 1028 can be a Fischer-Tropsch reactor for converting syngas in reactor feed stream 1026 to a Fischer-Tropsch product by conventional techniques as discussed above. For example, in a Fischer-Tropsch reaction, syngas composed of carbon monoxide (CO) and hydrogen gas (H₂), is converted in the presence of a Fischer-Tropsch catalyst (e.g., iron- or cobalt-based catalyst) into hydrocarbon products, water and other byproducts. The Fischer-Tropsch reactor can be any of those discussed above. The products produced from the reaction process in reactor unit 1028 can then be discharged from reactor unit 1028 as a reactor synthesis effluent 1030 comprising Fischer-Tropsch products.

In some embodiments, reactor unit 1028 can be a methanol and/or dimethyl ether reactor, rather than a Fischer-Tropsch reactor for processing reactor feed stream 1026. The products produced from the reaction process in reactor unit 1028 can then be discharged from reactor unit 1028 as reactor synthesis effluent 1030 comprising one or more of methanol and/or dimethyl ether.

In some embodiments, reactor unit 1028 can be a hydrogenation reactor for the direct hydrogenation of reactor feed stream 1026 to produce liquid hydrocarbons as discussed above.

In some embodiments, reactor feed stream 1026 can be subjected to direct hydrogenation of CO₂ to liquid hydrocarbons as discussed above.

Accordingly, for purposes of this illustrative embodiment, reactor synthesis effluent 1030 can be one or more of Fischer-Tropsch products, one or more of methanol and/or dimethyl ether products and direct hydrogenation products as well as unconverted feeds and by-products such as methane. However, this is merely illustrative and any other product that can be made from the conversion of reactor feed stream 1026 is contemplated herein for use as reactor synthesis effluent 1030.

The Fischer-Tropsch products, methanol/dimethyl ether products and/or direct hydrogenation products produced from the reaction process in reactor synthesis effluent 1030 can then be discharged from reactor unit 1028 and sent to separation unit 1032. Separation unit 1032 can be any of those discussed above for separation unit 122. For example, the Fischer-Tropsch products, methanol/dimethyl ether products and/or direct hydrogenation products can be separated and collected following conventional fractional distillation to produce a product effluent 1036 for sending further downstream for processing, while a portion of any unreacted syngas can be recycled to reactor unit 1028 for further conversion (not shown), and any methane and light hydrocarbons such as C₁ to C₄ light ends produced from the synthesis reactions can be recycled from separation unit 1032 as recycled tail gas stream 1014 back to cathode 1018-1 of second electrolyzer 1018 (see FIGS. 6A and 6B) or sent for further processing or reused as, for example, a fuel gas (see FIG. 6C) as discussed above. A liquid stream 1034 including at least hydrocarbons and water is sent for further downstream processing as known in the art. For example, product effluent 1036 can be sent for further downstream processing for creating high value liquid fuels, such as gasoline, diesel, and jet, or base oils.

In some embodiments, system 1000 may include a hydrocracker unit and/or fractionation unit to upgrade the Fischer-Tropsch liquids as discussed above for system 100.

In one or more illustrative embodiments, a system processing environment 1100 comprises each of the components of system 1000 described herein, as well as a controller 1110 operatively coupled to system processing environment 1100. Controller 1110 is configured to control operations of one or more of the components of system 1000 discussed above. In one illustrative embodiment, controller 1110 is configured to actuate one or more of the functionalities of system 1000 described herein. For example, controller 1110 can be similar to controller 210 described above and function in a similar manner.

The following illustrative examples are intended to be non-limiting.

### Examples 1 and 2 and Comparative Example A

The improvement of the energy efficiency obtained by recycling a C₁-C₄ tail gas stream to the cathode of a steam electrolyzer, where steam methane reforming takes place, was evaluated via process simulations. Example 1 is illustrative of the method and system of FIG. 1 and Example 2 is illustrative of the method and system of FIG. 3. The key performance indicators are summarized in Table 3. The target product for Examples 1 and 2 is a Synthetic Aviation Fuel (SAF).

TABLE 3

| | Comp. Ex. A | | |
|---|---|---|---|
| | Base Case | | |
| | (no tail gas recycle) | Example 1 | Example 2 |
| Total H₂O feed to SOEC, | 6.18E4 | 5.56E4 | 5.56E4 |
| lbmol/hr | | | (30% to steam feed |
| | | | stream 412) |
| | | | (70% to steam feed |
| | | | stream 402) |
| H₂ in SOEC outlet, | 4.93E4 | 4.93E4 | 4.93E4 |
| lbmol/hr | | | (30% to steam feed |
| | | | stream 412) |
| | | | (70% to steam feed |
| | | | stream 402) |
| CH₄ feed to SOEC, | — | 1883 | 1883 (to second |
| lbmol/hr | | | electrolyzer 418) |
| CH₄ remained in | — | 58 | 59.5 (in SOEC 418) |
| SOEC outlet, | | | |
| lbmol/hr | | | |
| CH₄ conversion % | — | 96.9 | 96.8 |
| SOEC power | 1524 | 1392 | 1391 by first |
| consumption, MW | | | electrolyzer 404, |
| | | | second |
| | | | electrolyzer 418 |
| H₂O conversion | 80 | 76 | 78 by first |
| in SOEC, % | | | electrolyzer 404, |
| | | | second |
| | | | electrolyzer 418 |
| CO₂ conversion % (to C₄₊)* | 84.4 | 95.6 | 95.3 |
| Energy Consumption, | 33.8 | 30.9 | 30.8 |
| kWh/kg H₂ | | | |

*The CO₂ conversion % (to C₄₊) is defined as the conversion rate of total CO₂ feed in the process to C₄₊ liquid fuel. The calculation is based on material balance only (i.e., assuming perfect separation of unreacted CO₂ and C₁₋₄ from C₄₊liquid products).

### Discussion of Example 1

The simulation for Example 1 investigated the effect of recycling all C₁ and C₂ (ethane/ethylene) from a Fischer-Tropsch (FT) or direct hydrogenation product to a cathode of a steam electrolyzer. The products from a FT or a direct hydrogenation reaction include both liquid and gaseous products from C₁ to C₂₃, where C₁ and C₂ constitute up to 10% of the total converted CO₂. These recycled species are reformed at the cathode of the SOEC (electrolyzer 110) for a target process of approximately 12K barrels per day (BPD) of Synthetic Aviation Fuel (SAF). For the SOEC process simulation, methane (C₁) was used as a model compound for reforming all light hydrocarbons ranging from C₁ to C₄ to simplify the model while still providing a reasonable approximation for analysis.

The key results set forth above in Table 3 from simulating recycling a tail gas stream to a cathode of a steam electrolyzer for Example 1 are:

1. Efficient Light End C₁-C₄ Hydrocarbon Reforming:

The simulation showed a 96.9% CH₄ conversion at the steam SOEC (electrolyzer 110) cathode inlet with an H₂O:CH₄ ratio of 30:1 and an average cell temperature of 741° C. This high conversion resulted in less than 0.055% of CH₄ within the reactor feed stream 116 (H₂+CO₂) to the FT reactor unit (reactor unit 118). Along SMR, which produced syngas (H₂+CO), the water gas shift (WGS) reaction occurs, resulting in CO₂ formation at its concentration equilibrium. Nonetheless, the overall H₂:C (CO+CO₂) ratio resulted from SMR and corresponding WGS reaction was 3:1, consistent with the feed requirement of FT or direct hydrogenation (H₂:CO₂=3:1) in reactor feed stream 116.

### 2. High Energy Efficiency, Smaller Cell Area and Lower Steam Feed Demand:

Due to the 96.9% conversion of CH₄ to syngas (H₂+CO) in cathode effluent 114, a 10% reduction in steam feed demand in cathode feed stream 104 for electrolysis in SOEC (electrolyzer 110) was achieved. The produced syngas via SMR also lowered the CO₂ demand in carbon dioxide feed stream 108 for the FT synthesis in reactor unit 118, which in turn boosted the CO₂ conversion to valuable C₄₊ liquid products to 95.5%, comparing to 84.4% for base case with no reforming. A 13% less cell area was needed for the process, resulting in a smaller process footprint and CAPEX (Capital Expenses). The energy demand of the process is measured in kilowatt-hours per kilogram of Hydrogen (kWh/kg H₂) to quantify the amount of electrical energy required to produce a certain amount of hydrogen used as a key component in the production of SAF. Thus, the lower this value, the more energy-efficient the process. Correspondingly, the energy demand of steam electrolysis per unit SAF produced was reduced in the SOEC, leading to an 8.7% increase in energy efficiency. For example, the energy consumption by the SOEC was 30.9 kWh/kg H₂ with CH₄ recycle, compared to the base case energy consumption of 33.8 kWh/kg H₂ without CH₄ recycle.

In conclusion, recycling a tail gas stream and reforming CH₄ in the cathode SOEC is believed to improve the overall energy efficiency. In addition, recycling a tail gas stream and reforming CH₄ in the cathode SOEC offers several other advantages, including a smaller steam demand, higher CO₂ utilization, and the elimination of C₁ content build-up in the FT reaction, without altering the FT H₂:carbon feed ratio.

### Discussion of Example 2

The simulation for Example 2 investigated the effect of recycling all C₁ and C₂ (ethane/ethylene) from a Fischer-Tropsch (FT) or direct hydrogenation product to a separate steam electrolyzer SOEC (second electrolyzer 418). The products from a FT or a direct hydrogenation reaction include both liquid and gaseous products from C₁ to C₂₃, where C₁ and C₂ constitute up to 10% of the total converted carbon. These recycled species are reformed at the SOEC (second electrolyzer 418) cathode 418-1, from which the cathode effluent, combined with syngas stream (cathode effluent 408) produced from SOEC (first electrolyzer 404), are fed to a target process, aiming approximately 12K BPD of SAF. For SOEC process simulation, methane (CH₄) was used as a model compound for reforming all light hydrocarbons ranging from C₁ to C₄ to simplify the model while still providing a reasonable approximation for analysis.

In this simulation, the same total steam feed was applied as in Example 1, where 30% of a steam feed was charged to the SOEC (second electrolyzer 418).

The key results set forth above in Table 1 from simulating recycling a tail gas stream to a cathode of a steam electrolyzer for Example 2 are:

### 1. Efficient Hydrocarbon Reforming at SOEC Cathode:

The simulation showed a 96.8% CH₄ conversion at SOEC (second electrolyzer 418) cathode inlet with an H₂O:CH₄ ratio of 9:1 and an average cell temperature of 698° C. This high conversion results in less than 0.055% of CH₄ content in the overall feed (reactor feed stream 426) to the FT reactor (reactor unit 428). Along SMR, which produced the syngas (H₂+CO), the WGS reaction also occurs, resulting in CO₂ formation at its reaction equilibrium. In addition, SOEC (second electrolyzer 418) provides 30% of total H₂ required for the target FT process in reactor unit 428.

### 2. High Energy Efficiency and Smaller Cell Area:

Compared to Example 1, reforming tail gas at the SOEC (second electrolyzer 418) led to an energy consumption by Example 2 of 30.8 kWh/kg H₂ as compared to the base case energy consumption of 33.9 kWh/kg H₂. Demonstrating a similar CH₄ conversion rate as Example 1, Example 2 reached a similar CO₂ conversion to valuable C₄₊ liquid products of 95.3%. A 4% less total cell area is required by Example 2, resulting in a smaller process footprint and CAPEX.

In conclusion, recycling C₁ in the cathode of the SOEC (second electrolyzer 418) with 30% of total steam feed is believed to improve the overall energy efficiency.

### Examples 3-5 and Comparative Example B

Example 3 is illustrative of the method and system of FIG. 4. Example 4 is illustrative of the method and system of FIG. 5. Example 5 is illustrative of the method and system of FIG. 6. The target product for Examples 3-5 is a base oil.

In Example 3, the improvement of the energy efficiency obtained by recycling tail gas to the cathode of the electrolyzer, where steam and CO₂ react via co-electrolysis and SMR occurs, was evaluated through process simulation. In Example 4, the energy efficiency scenario was evaluated via process simulation and benchmarked against Example 3. In Example 5, the energy efficiency scenario was evaluated via process simulation and benchmarked against Examples 3 and 4.

The key performance indicators are summarized in Table 4.

TABLE 4

| | Comp. Ex. B | | | |
|---|---|---|---|---|
| | Base Case | | | |
| | (no C₁ recycle) | Example 3 | Example 4 | Example 5 |
| H₂O feed to co- | 7.54E4 | 7.3E4 | 7.3E4 | 7.3E4 |
| SOEC¹, | | (electrolyzer 608) | (30% to steam and | (23% to steam feed |
| lbmol/hr | | | carbon dioxide feed | stream 1012) |
| | | | stream 809) | (77% to steam and |
| | | | (70% to steam and | carbon dioxide feed |
| | | | carbon dioxide feed | stream 1002) |
| | | | stream 802) | |
| CO₂ feed to co- | 2.60E4 | 2.44E4 | 2.44E4 | 2.44E4 |
| SOEC, lbmol/hr | | (electrolyzer 608) | (30% to steam and | (100% to first |
| | | | carbon dioxide feed | electrolyzer 1004) |
| | | | stream 809) | |
| | | | (70% to steam and | |
| | | | carbon dioxide feed | |
| | | | stream 802) | |
| H₂ in co-SOEC | 6.17E4 | 6.17E4 | 6.17E4 | 6.17E4 |
| outlet, lbmol/hr | | (electrolyzer608) | (34% to steam and | (26% to steam feed |
| | | | carbon dioxide feed | stream 1012) |
| | | | stream 809) | (74% to steam and |
| | | | (66% to steam and | carbon dioxide feed |
| | | | carbon dioxide feed | stream 1002) |
| | | | stream 802) | |
| CO in co-SOEC | 2.00E4 | 2.00E4 | 2.00E4 | 2.00E4 |
| outlet, lbmol/hr | | (electrolyzer 608) | (36% to steam and | (6% to steam feed |
| | | | carbon dioxide feed | stream 1012) |
| | | | stream 809) | (94% to steam and |
| | | | (64% to steam and | carbon dioxide feed |
| | | | carbon dioxide feed | stream 1002) |
| | | | stream 802) | |
| CH₄ feed to co- | — | 2203 (elec- | 2203 (in | 2203 (in |
| SOEC, lbmol/hr | | trolyzer608) | second | second |
| | | | electrolyzer 814) | Electrolyzer 1018) |
| H₂O:CH₄ feed | — | 38.77 (elec- | 9:1 (in | 9:1 (in |
| ratio to co- | | trolyzer 608) | second | second |
| SOEC | | | electrolyzer 814) | electrolyzer 1018) |
| CH₄ remained | — | 308 (in | 311 (62 in | 346 (40 by |
| in co-SOEC | | cathode | second | second |
| outlet, lbmol/hr | | effluent 612) | electrolyzer 814 | electrolyzer 1018 |
| | | | 249 by methanation | 306 by methanation |
| | | | in first electrolyzer | in first electrolyzer |
| | | | 804) | 1004) |
| CH₄ content in | — | 0.24 (in | 0.32 (in | 0.39 (in |
| feed to Fischer- | | cathode | reactor feed | reactor feed |
| Tropsch (FT), | | effluent 612) | stream 820) | stream 1026) |
| % | | | | |
| CH₄ | — | 86 (elec- | 97.2 (in | 98.2 (in |
| conversion % | | trolyzer608) | second | second |
| via SMR | | | electrolyzer 814) | electrolyzer 1018) |
| H₂:C (CO + CO₂) | 2.4:1 | 2.4:1 (in | 2.4:1 (in | 2.4:1 (in |
| feed to FT | | cathode | reactor feed | reactor feed |
| | | effluent 612) | stream 820) | stream 1026) |
| Co-SOEC | 2630 | 2465 | 2342 | 2490 |
| power | | | | |
| consumption, | | | | |
| MW | | | | |
| Steam | 82 | 78 | 77 | 77 |
| electrolysis, | | | | |
| conversion % | | | | |
| CO₂ | 77 | 75 | 74 | 74 |
| electrolysis, | | | | |
| conversion % | | | | |
| CO₂ conversion | 88.8 | 94.8 | 94.8 | 94.8 |
| % (to C₄₊)² | | | | |
| Energy | 3.16 | 2.96 | 2.81 | 2.98 |
| Consumption, | | | | |
| kWh/Nm³ | | | | |
| (H₂:CO = 3:1) | | | | |

¹Co-electrolysis electrolyzer (co-SOEC).
²The CO₂ conversion % (to C₄₊) is defined as the conversion rate of total CO₂ feed in the process to C₄₊ liquid fuel. The calculation is based on material balance only (i.e., assuming perfect separation of unreacted CO₂ and C₁₋₄ from C₄₊liquid products).

### Discussion of Example 3

The simulation for Example 3 investigated the effect of recycling all C₁ and C₂ (ethane/ethylene) from the FT product to the cathode 608-1 of co-SOEC (electrolyzer 608). In this scenario, the FT products include both liquid and gaseous products from C₁ to C₃₅ and C₁ and C₂ constitute up to 8.5% of the total converted carbon. These recycled species are reformed at the co-SOEC (electrolyzer 608) cathode for a target process of approximately 12K BPD of Base Oil production via a syngas to FT conversion process. For SOEC process simulation, methane (C₁) was used as a model compound for reforming all light hydrocarbons ranging from C₁ to C₄ to simplify the model for analysis.

The key results set forth above in Table 2 from simulating recycling a tail gas stream to a cathode of a co-SOEC for Example 3 are:

### 1. Efficient Hydrocarbon Reforming:

The simulation demonstrates an 86% CH₄ conversion at the co-SOEC (electrolyzer 608) cathode inlet with a H₂O:CH₄ ratio of ˜39:1 and an average cell temperature of ˜762° C. This high conversion results in less than 0.24% of CH₄ within the overall syngas feed stream (cathode effluent 612) to the FT reaction.

### 2. High Energy Efficiency, Smaller Cell Area and Lower Steam Feed Demand:

The 86% conversion of CH₄ to syngas (H₂+CO) results in greater than 3% and 6.5% reduction in steam and CO₂ feed demand to co-SOEC (electrolyzer 608), respectively, for co-electrolysis to achieve the same syngas required for producing 12K BPD Base Oil production as the base case. The lowered CO₂ demand for the FT synthesis in reactor unit 614 in turn boosted the CO₂ conversion to valuable C₄₊ liquid products to 94.8%, comparing to 88.8% by base case. A 6.3% less cell area was needed for the process, encouraging a smaller process footprint and CAPEX. This reduces the energy demand of co-electrolysis in SOEC, leading to a 6.3% increase in energy efficiency. For example, the energy consumption by co-SOEC (electrolyzer 608) was 2.96 kWh/Nm³ (H₂:CO=3:1) with CH₄ recycle, compared to the base case energy consumption of 3.16 kWh/Nm³ (H₂:CO=3:1) without CH₄ recycle. Although the required H₂:CO ratio in cathode effluent 612 for the syngas-to-FT process was 2:1, the remaining 25% unreacted CO₂ from the electrolysis process was co-fed to the FT reactor, where CO₂ was assumed to react via direct hydrogenation. Therefore, a higher H₂ content as 3:1 H₂/CO feed ratio (or H₂:C(CO+CO₂) of 2.4:1) in SOEC (electrolyzer 608) was implemented for energy efficiency calculation.

In conclusion, recycling a tail gas stream and reforming C₁ at the cathode SOEC is believed to enhance the overall energy efficiency and carbon conversion efficiency. It also offers several other benefits, including reduced steam and CO₂ demand, smaller cell area, and the prevention of tail gas build-up in the FT step.

### Discussion of Example 4

The simulation for Example 4 investigated the energy efficiency and benchmarked against Example 3. In both cases, the H₂O and CO₂ feed quantities and CO and H₂ product amounts were implemented to target 12K BPD base oil production. Specifically in Example 4, SOEC (second electrolyzer 814) takes up 30% total H₂O and CO₂ feed of the overall process, where steam and CO₂ co-electrolysis and SMR occur at cathode 814-1. For SOEC process simulations, methane (C₁) was used as a model compound for reforming all light hydrocarbons ranging from C₁ to C₄ to simplify the model for analysis.

The key results set forth above in Table 4 from simulating recycling a tail gas stream to a cathode of SOEC 2 for Example 4 are:

### 1. Efficient Hydrocarbon Reforming:

The simulation demonstrates a 97% CH₄ conversion at a SOEC (second electrolyzer 814) cathode inlet with H₂O:CH₄ ratio of ˜9:1 and an average cell temperature of approximately 737° C. However, it was observed that there was an additional 250 lbmol/hr of CH₄ formed at SOEC (first electrolyzer 804) due to methanation, which brought the net CH₄ conversion rate to 84%, which was close to that of Example 3. Nonetheless, the combined CH₄ took up trace (0.32%) of the overall syngas feed stream (reactor feed stream 820) to the FT reaction in reactor unit 822. In demonstrating a similar total CH₄ conversion rate as Example 3, Example 4 reached a similar CO₂ conversion to valuable C₄₊ liquid products of 94.8%.

### 2. High Energy Efficiency and Less Cell Area:

Example 4 demonstrated 5% lower energy consumption than Example 3, i.e., the energy consumption for Example 4 was 2.81 kWh/Nm³ (H₂:CO=3:1), compared to 2.96 kWh/Nm³ (H₂:CO=3:1) for Example 3. It was observed that the lower H₂O and CO₂ conversion (77% and 74%, respectively) via electrolysis in SOEC (second electrolyzer 814) than SOEC (first electrolyzer 804) (78% and 75%, respectively) contributed to the lower averaged energy consumption than Example 3. Specifically, co-SOEC 2 consumed 25% of total energy to generate 34% H₂O and 36% CO needed by the process. On the other hand, a 4% less total cell area was needed for Example 4, resulting in a smaller process footprint and CAPEX. Although the required H₂:CO ratio for the syngas-to-FT process was 2:1, the remaining 25% unreacted CO₂ from the electrolysis process was co-fed to the FT reactor, where CO₂ was assumed to react via direct hydrogenation. Therefore, a higher H₂ content of 3:1 H₂:CO feed ratio (or H₂:C(CO+CO₂) of 2.4:1) was implemented for energy efficiency calculation.

In conclusion, recycling of a tail gas stream and reforming CH₄ at a smaller dedicated cathode SOEC 2 is believed to enhance the overall energy efficiency. It also offers several other benefits, such as smaller cell area and more flexible operation.

### Discussion of Example 5

The simulation for Example 5 investigated the energy efficiency and benchmarked against Examples 3 and 4. In each case, the same H₂O and CO₂ feed quantities and CO and H₂ product target quantities were implemented to target 12K BPD base oil production. Specifically, SOEC (second electrolyzer 1018), where steam electrolysis and SMR occur, takes up to 23% total H₂O feed to the process. On the other hand, SOEC (first electrolyzer 1004) takes up the rest 77% steam and 100% CO₂ of the process. In Example 5, the recycled tail gas was reformed at the SOEC (second electrolyzer 1018) cathode, from which the syngas product will combine with SOEC (first electrolyzer 1004) syngas product stream in reactor feed stream 1026 to be charged to the FT conversion process. For SOEC process simulations, methane (C₁) was used as a model compound for reforming all light hydrocarbons ranging from C₁ to C₄ to simplify the model for analysis.

The key results set forth above in Table 4 from simulating recycling a tail gas stream to a cathode of SOEC 2 for Example 5 are:

### 1. Efficient Hydrocarbon Reforming:

The simulation demonstrates a 98% CH₄ conversion at a SOEC (second electrolyzer 1018) cathode inlet with H₂O:CH₄ ratio of ˜9:1 and an average cell temperature of ˜702° C. However, it was observed that there was an additional 306 lbmol/hr of CH₄ formed at SOEC (first electrolyzer 1004) due to methanation, which brought the net C1 conversion rate to 82%. Nonetheless, the combined CH₄ takes up trace (0.39%) of the overall syngas feed in reactor feed stream 1026 to the FT reaction in reactor unit 1028. In demonstrating a similar total CH₄ conversion rate as Examples 3 and 4, Example 5 reached a similar CO₂ conversion to valuable C₄₊ liquid products of 94.8%.

### 2. High Energy Efficiency and Less Cell Area:

Example 5 demonstrated an energy consumption of 2.98 kWh/Nm³ (H₂:CO=3:1). Among the total energy required, 85% of total power consumption was associated with SOEC (first electrolyzer 1004), due to its need of 96% CO production for the overall process. When compared to Example 3, where tail gas was recycled and reformed in the single SOEC (second electrolyzer 1018), Example 5 showed a slight increase in energy consumption by 0.7%. Although the required H₂:CO ratio for the syngas-to-FT process was 2:1, the remaining 25% unreacted CO₂ from the electrolysis process is co-fed to the FT reactor, where CO₂ was assumed to react via direct hydrogenation. Therefore, a higher H₂ content as 3:1 H₂/CO feed ratio (or H₂:C(CO+CO₂) of 2.4:1) was implemented for energy efficiency calculation.

In conclusion, recycling of a tail gas stream and reforming C₁ at a smaller dedicated cathode SOEC (second electrolyzer 1018) is believed to minimize C₁ to C₂ tail gas components from the process. Although it had the highest energy consumption among each of Examples 3-5, it is still lower than that of base case and offers key advantage of flexible operation of the dedicated steam electrolyzer.

### Example 6 and Comparative Example C

Example 6 is illustrative of the method and system of FIG. 4.

The key performance indicators are summarized in Table 5.

TABLE 5

| | Comp. Ex. C | |
|---|---|---|
| | Base Case | |
| | (no C₁ feed) | Example 6 |
| H₂O feed (kmol/hr) | 6300 | 5177 |
| CO₂ feed (kmol/hr) | 2114 | 1609 |
| CH₄ feed (kmol/hr) | — | 536 |
| H₂O conv. in SOEC | 82% | 89% |
| CO₂ conv. in SOEC | 78% | 81% |
| CH₄ conv. in SOEC | — | 97% |
| SOEC power consumption, | 484 | 356 |
| (MW) | | |
| H₂:C (CO + CO₂) feed ratio | 2.46:1 | 2.27:1 |
| to Methanol reactor | | |
| Feed Pressure, Temperature | 60 bar/180 C. | 60 bar/180 C. |
| to Methanol Reactor | | |
| (i.e., Stream 612) | | |
| MeOH Production | 12 kBPD | 12 kBPD |
| Carbon (CO₂ and CH₄) | 95% | 93% |
| conversion rate to MeOH ¹ | | |

¹ MeOH conversion relative to total carbon (or CO₂ + CH₄) feed

The energy efficiency improvement achieved by introducing methane/natural gas/renewable natural gas to the cathode of a co-electrolyzer, where co-electrolysis of CO₂ and steam, along with steam methane reforming reaction, was evaluated through process simulations. Example 6 illustrates the method and system of FIG. 4C.

### Discussion of Example 6

The simulation for Example 6 explored the impact of introducing methane/natural gas/renewable natural gas to a cathode of a co-electrolyzer. The CH₄ component undergoes reforming at the cathode 608-1 of the SOEC (electrolyzer 608), in conjunction with co-electrolysis, to produce syngas targeted for 12K BPD of methanol production.

For the SOEC process simulation, methane was used as a representative compound for reforming the introduced natural gas/renewable natural gas in methane-rich feed stream 611, where methane to CO₂ ratio in methane-rich feed stream 611 is 1:4. This simplifies the model while still providing a reasonable approximation for analysis.

The key results from simulating the reforming of methane/natural gas/renewable natural gas at a cathode of a steam electrolyzer for producing 12K BPD methanol for Example 6 are as follows:

### 1. Efficient C₁ Reforming:

The simulation demonstrated a 97% CH₄ conversion of the methane-rich feed stream 611 in SOEC (electrolyzer 608). Alongside SMR, which produced syngas (H₂+CO), the water gas shift (WGS) reaction occurred, resulting in CO₂ formation at its concentration equilibrium. However, the overall H2:C (CO+CO₂) ratio resulting from SMR and the corresponding WGS reaction was approximately 2.3:1, consistent with the feed requirement of syngas to FT synthesis (H₂:CO₂=2:1) in stream (cathode effluent 612).

### 2. High Energy Efficiency and Lower Steam and CO2 Feed Demand:

Due to the 97% conversion of CH₄ to syngas (H₂+CO) in SOEC (electrolyzer 608), there was an 18% reduction in steam feed and a 24% reduction in CO₂ feed in steam and carbon dioxide feed stream 602 for co-electrolysis in SOEC (electrolyzer 608), compared to the base case, for the same 12K BPD methanol synthesis. Consequently, the energy demand by electrolyzer 608 in Example 6 with methane recycle showed an enhancement in energy efficiency by consuming 26% less power. For instance, the energy consumption by the SOEC in Example 6 was 356 MW with CH₄ recycle, compared to the base case energy consumption of 484 MW without CH₄ recycle.

Reforming CH₄/natural gas/renewable natural gas in the cathode SOEC is believed to improve the overall energy efficiency, and it offers several other advantages, including smaller feed demand to SOEC, and feedstock flexibility for producing methanol.

According to an aspect of the present disclosure, a continuous process comprises:
sending one or more of a recycled tail gas stream from a reactor unit and a methane-rich feed stream, and one of a steam feed stream or a steam and carbon dioxide feed stream to a cathode of an electrolyzer comprising the cathode, an anode and an electrolyte inserted between the cathode and the anode to produce a cathode effluent comprising a first syngas,
sending a reactor unit product-forming feed stream comprising the cathode effluent to the reactor unit, wherein the reactor unit product-forming feed stream includes, in addition to the cathode effluent, at least one of a second syngas and carbon dioxide, and
generating, in the reactor unit, a chemical product or a fuel based, in part, on the reactor unit product-forming feed stream.

According to another aspect of the present disclosure, a system comprises:
an electrolyzer comprising a cathode, an anode and an electrolyte inserted between the cathode and the anode, wherein the electrolyzer is configured to receive one or more of a recycled tail gas stream from a reactor unit and a methane-rich feed stream, and one of a steam feed stream or a steam and carbon dioxide feed stream to the cathode to produce a cathode effluent comprising a first syngas,
wherein the reactor unit is configured to receive a reactor unit product-forming feed stream comprising the cathode effluent, wherein the reactor unit product-forming feed stream includes, in addition to the cathode effluent, at least one of a second syngas and carbon dioxide, and produce a chemical product or a fuel based, in part, on the reactor unit product-forming feed stream.

According to another aspect of the present disclosure, a continuous process comprises:
generating, in an electrolyzer, a cathode effluent comprising syngas derived from one or more of a recycled tail gas stream from a reactor unit and a methane-rich feed stream,
sending the cathode effluent and a carbon dioxide feed stream to the reactor unit, and
processing, in the reactor unit, the first cathode effluent and the carbon dioxide feed stream to produce a chemical product or a fuel base.

According to another aspect of the present disclosure, a system comprises:
an electrolyzer comprising a cathode, an anode and an electrolyte inserted between the cathode and the anode, wherein the electrolyzer is configured to receive a steam feed stream and one or more of a recycled tail gas stream from a reactor unit and a methane-rich feed stream to the cathode to produce a cathode effluent comprising syngas,
wherein the reactor unit is configured to receive the cathode effluent and a carbon dioxide feed stream, and produce a chemical product or a fuel base.

According to another aspect of the present disclosure, a continuous process comprises:
generating, in a first electrolyzer, a first cathode effluent comprising hydrogen,
generating, in a second electrolyzer, a second cathode effluent comprising syngas derived from one or more of a recycled tail gas stream from a reactor unit and a methane-rich feed stream,
sending the first cathode effluent, the second cathode effluent and a carbon dioxide feed stream to the reactor unit, and
processing, in the reactor unit, the first cathode effluent, the second cathode effluent and the carbon dioxide feed stream to produce a chemical product or a fuel base.

According to another aspect of the present disclosure, a system comprises:
a first electrolyzer comprising a first cathode, a first anode and a first electrolyte inserted between the first cathode and the first anode, wherein the first electrolyzer is configured to receive a first steam feed stream to the cathode to produce a first cathode effluent comprising hydrogen, and
a second electrolyzer comprising a second cathode, a second anode and a second electrolyte inserted between the second cathode and the second anode, wherein the second electrolyzer is configured to receive a second steam feed stream and one or more of a recycled tail gas stream from a reactor unit and a methane-rich feed stream to the cathode to produce a second cathode effluent comprising syngas,
wherein the reactor unit is configured to receive the first cathode effluent, the second cathode effluent and a carbon dioxide feed stream and produce a chemical product or a fuel base.

According to another aspect of the present disclosure, a continuous process comprises:
passing a steam and carbon dioxide steam feed and one or more of a recycled tail gas stream from a reactor unit and a methane-rich feed stream to a cathode of an electrolyzer,
generating a cathode effluent comprising carbon monoxide, hydrogen, carbon dioxide and methane based, in part, on the steam and carbon dioxide steam feed and the one or more of the recycled tail gas stream and the methane-rich feed stream, and
processing, in the reactor unit, the cathode effluent to produce a chemical product or a fuel base.

According to another aspect of the present disclosure, a system comprises:
an electrolyzer comprising a cathode, an anode and an electrolyte inserted between the cathode and the anode, wherein the electrolyzer is configured to receive a steam and carbon dioxide feed stream and one or more of a recycled tail gas stream from a reactor unit and a methane-rich feed stream to the cathode to produce a cathode effluent comprising carbon monoxide, hydrogen, carbon dioxide and methane,
wherein the reactor unit is configured to receive the cathode effluent and produce a chemical product or a fuel base.

According to another aspect of the present disclosure, a continuous process comprises:
generating, in a first electrolyzer, a first cathode effluent comprising syngas,
generating, in a second electrolyzer, a second cathode effluent comprising carbon monoxide, hydrogen, carbon dioxide and methane derived from a steam and carbon dioxide feed stream and one or more of a recycled tail gas stream from a reactor unit and a methane-rich feed stream,
sending the first cathode effluent and the second cathode effluent to the reactor unit, and
processing, in the reactor unit, the first cathode effluent and the second cathode effluent to produce a chemical product or a fuel base.

According to another aspect of the present disclosure, a system comprises:
a first electrolyzer comprising a first cathode, a first anode and a first electrolyte inserted between the first cathode and the first anode, wherein the first electrolyzer is configured to receive a first steam and carbon dioxide feed stream to the cathode to produce a first cathode effluent comprising syngas, and
a second electrolyzer comprising a second cathode, a second anode and a second electrolyte inserted between the second cathode and the second anode, wherein the second electrolyzer is configured to receive a second steam and carbon dioxide feed stream and one or more of a recycled tail gas stream from a reactor unit and a methane-rich feed stream to the cathode to produce a second cathode effluent comprising carbon monoxide, hydrogen, carbon dioxide and methane,
wherein the reactor unit is configured to receive the first cathode effluent and the second cathode effluent and produce a chemical product or a fuel base.

According to another aspect of the present disclosure, a continuous process comprises:
generating, in a first electrolyzer, a first cathode effluent comprising syngas,
generating, in a second electrolyzer, a second cathode effluent comprising syngas derived from a steam feed stream and one or more of a recycled tail gas stream from a reactor unit and a methane-rich feed stream,
sending the first cathode effluent and the second cathode effluent to the reactor unit, and
processing, in the reactor unit, the first cathode effluent and the second cathode effluent to produce a chemical product or a fuel base.

According to another aspect of the present disclosure, a system comprises:
a first electrolyzer comprising a first cathode, a first anode and a first electrolyte inserted between the first cathode and the first anode, wherein the first electrolyzer is configured to receive a steam and carbon dioxide feed stream to the cathode to produce a first cathode effluent comprising syngas, and
a second electrolyzer comprising a second cathode, a second anode and a second electrolyte inserted between the second cathode and the second anode, wherein the second electrolyzer is configured to receive a steam feed stream and one or more of a recycled tail gas stream from a reactor unit and a methane-rich feed stream to the cathode to produce a second cathode effluent comprising syngas,
wherein the reactor unit is configured to receive the first cathode effluent and the second cathode effluent and produce a chemical product or a fuel based.

According to another aspect of the present disclosure, a continuous method comprises:
passing a first steam feed stream and one or more of a recycled tail gas stream and a methane-rich feed stream to a cathode of a first electrolyzer comprising the cathode, an anode and an electrolyte inserted between the cathode and the anode, thereby producing a cathode effluent comprising syngas, and
passing the cathode effluent comprising syngas to a reactor unit, thereby producing a chemical product or a fuel-based product.

In one or more additional illustrative embodiments, as may be combined with the preceding paragraphs, the continuous method further comprises passing the cathode effluent comprising syngas with a carbon dioxide feed stream to the reactor unit, thereby producing the chemical product or the fuel-based product.

In one or more additional illustrative embodiments, as may be combined with the preceding paragraphs, the chemical product is one or more of methanol and dimethyl ether.

In one or more additional illustrative embodiments, as may be combined with the preceding paragraphs, the fuel-based product is a Fischer-Tropsch product.

In one or more additional illustrative embodiments, as may be combined with the preceding paragraphs, the continuous method further comprises:
passing a second steam feed stream to a cathode of a second electrolyzer comprising the cathode, an anode and an electrolyte inserted between the cathode and the anode, thereby producing a cathode effluent comprising hydrogen,
passing the cathode effluent from the first electrolyzer, the cathode effluent from the second electrolyzer and a carbon dioxide feed stream to the reactor unit, and
processing, in the reactor unit, the cathode effluent from the first electrolyzer, the cathode effluent from the second electrolyzer and the carbon dioxide feed stream, thereby producing the chemical product or the fuel-based product.

In one or more additional illustrative embodiments, as may be combined with the preceding paragraphs, the chemical product is one or more of methanol and dimethyl ether.

In one or more additional illustrative embodiments, as may be combined with the preceding paragraphs, the fuel-based product is a Fischer-Tropsch product.

In one or more additional illustrative embodiments, as may be combined with the preceding paragraphs, the continuous method further comprises:
passing a steam and carbon dioxide feed stream to a cathode of a second electrolyzer comprising the cathode, an anode and an electrolyte inserted between the cathode and the anode, thereby producing a cathode effluent comprising syngas,
passing the cathode effluent from the first electrolyzer and the cathode effluent from the second electrolyzer to the reactor unit, and
processing, in the reactor unit, the cathode effluent from the first electrolyzer and the cathode effluent from the second electrolyzer, thereby producing the chemical product or the fuel-based product.

In one or more additional illustrative embodiments, as may be combined with the preceding paragraphs, the second electrolyzer is configured to operate in a co-electrolysis mode.

In one or more additional illustrative embodiments, as may be combined with the preceding paragraphs, the chemical product is one or more of methanol and dimethyl ether.

In one or more additional illustrative embodiments, as may be combined with the preceding paragraphs, the fuel-based product is a Fischer-Tropsch product.

In one or more additional illustrative embodiments, as may be combined with the preceding paragraphs, the continuous method comprises passing the first steam feed stream and the recycled tail gas stream to the cathode of the first electrolyzer, thereby producing the cathode effluent comprising syngas.

In one or more additional illustrative embodiments, as may be combined with the preceding paragraphs, the continuous method comprises passing the first steam feed stream and the methane-rich feed stream to the cathode of the first electrolyzer, thereby producing the cathode effluent comprising syngas.

In one or more additional illustrative embodiments, as may be combined with the preceding paragraphs, the continuous method comprises passing the first steam feed stream, the recycled tail gas stream and the methane-rich feed stream to the cathode of the first electrolyzer, thereby producing the cathode effluent comprising syngas.

In one or more additional illustrative embodiments, as may be combined with the preceding paragraphs, passing the cathode effluent comprising syngas to the reactor unit further produces a tail gas stream, and the continuous method further comprises:
recycling the tail gas stream to the cathode of the first electrolyzer.

According to another aspect of the present disclosure, a system comprises:
a first electrolyzer comprising a cathode, an anode and an electrolyte inserted between the cathode and the anode, wherein the first electrolyzer is configured to receive in the cathode a first steam feed stream and one or more of a recycled tail gas stream from a reactor unit and a methane-rich feed stream, thereby producing a cathode effluent comprising syngas, and
a reactor unit configured to receive the cathode effluent comprising syngas, thereby producing a chemical product or a fuel-based product.

In one or more additional illustrative embodiments, as may be combined with the preceding paragraphs, the system further comprises:
a carbon dioxide source for passing the reactor unit;
wherein the reactor unit is further configured to receive a carbon dioxide feed stream from the carbon dioxide source with the cathode effluent comprising syngas, thereby producing the chemical product or the fuel-based product.

In one or more additional illustrative embodiments, as may be combined with the preceding paragraphs, the system further comprises:
a second electrolyzer comprising a cathode, an anode and an electrolyte inserted between the cathode and the anode, wherein the second electrolyzer is configured to receive in the cathode a second steam feed stream, thereby producing a cathode effluent comprising hydrogen,
wherein the reactor unit is further configured to receive the cathode effluent from the second electrolyzer with the cathode effluent from the first electrolyzer for producing the chemical product or the fuel-based product.

In one or more additional illustrative embodiments, as may be combined with the preceding paragraphs, the system further comprises:
a second electrolyzer comprising a cathode, an anode and an electrolyte inserted between the cathode and the anode, wherein the second electrolyzer is configured to receive in the cathode a steam and carbon dioxide feed stream, thereby producing a cathode effluent comprising syngas,
wherein the reactor unit is further configured to receive the cathode effluent from the second electrolyzer with the cathode effluent from the first electrolyzer for producing the chemical product or the fuel-based product.

In one or more additional illustrative embodiments, as may be combined with the preceding paragraphs, the reactor unit is one of a Fischer-Tropsch reactor unit or a methanol and dimethyl ether reactor unit.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A continuous method, comprising:
passing a first steam feed stream and one or more of a recycled tail gas stream and a methane-rich feed stream to a cathode of a first electrolyzer comprising the cathode, an anode and an electrolyte inserted between the cathode and the anode, thereby producing a cathode effluent comprising syngas; and
passing the cathode effluent comprising syngas to a reactor unit, thereby producing a chemical product or a fuel-based product.

2. The continuous method according to claim 1, further comprising passing the cathode effluent comprising syngas with a carbon dioxide feed stream to the reactor unit, thereby producing the chemical product or the fuel-based product.

3. The continuous method according to claim 2, wherein the chemical product is one or more of methanol and dimethyl ether.

4. The continuous method according to claim 2, wherein the fuel-based product is a Fischer-Tropsch product.

5. The continuous method according to claim 1, further comprising:
passing a second steam feed stream to a cathode of a second electrolyzer comprising the cathode, an anode and an electrolyte inserted between the cathode and the anode, thereby producing a cathode effluent comprising hydrogen;
passing the cathode effluent from the first electrolyzer, the cathode effluent from the second electrolyzer and a carbon dioxide feed stream to the reactor unit; and
processing, in the reactor unit, the cathode effluent from the first electrolyzer, the cathode effluent from the second electrolyzer and the carbon dioxide feed stream, thereby producing the chemical product or the fuel-based product.

6. The continuous method according to claim 5, wherein the chemical product is one or more of methanol and dimethyl ether.

7. The continuous method according to claim 5, wherein the fuel-based product is a Fischer-Tropsch product.

8. The continuous method according to claim 1, further comprising:
passing a steam and carbon dioxide feed stream to a cathode of a second electrolyzer comprising the cathode, an anode and an electrolyte inserted between the cathode and the anode, thereby producing a cathode effluent comprising syngas;
passing the cathode effluent from the first electrolyzer and the cathode effluent from the second electrolyzer to the reactor unit; and
processing, in the reactor unit, the cathode effluent from the first electrolyzer and the cathode effluent from the second electrolyzer, thereby producing the chemical product or the fuel-based product.

9. The continuous method according to claim 8, wherein the second electrolyzer is configured to operate in a co-electrolysis mode.

10. The continuous method according to claim 8, wherein the chemical product is one or more of methanol and dimethyl ether.

11. The continuous method according to claim 8, wherein the fuel-based product is a Fischer-Tropsch product.

12. The continuous method according to claim 1, comprising passing the first steam feed stream and the recycled tail gas stream to the cathode of the first electrolyzer, thereby producing the cathode effluent comprising syngas.

13. The continuous method according to claim 1, comprising passing the first steam feed stream and the methane-rich feed stream to the cathode of the first electrolyzer, thereby producing the cathode effluent comprising syngas.

14. The continuous method according to claim 1, comprising passing the first steam feed stream, the recycled tail gas stream and the methane-rich feed stream to the cathode of the first electrolyzer, thereby producing the cathode effluent comprising syngas.

15. The continuous method according to claim 1, wherein passing the cathode effluent comprising syngas to the reactor unit further produces a tail gas stream, and the continuous method further comprises:
recycling the tail gas stream to the cathode of the first electrolyzer.

16. A system, comprising:
a first electrolyzer comprising a cathode, an anode and an electrolyte inserted between the cathode and the anode, wherein the first electrolyzer is configured to receive in the cathode a first steam feed stream and one or more of a recycled tail gas stream from a reactor unit and a methane-rich feed stream, thereby producing a cathode effluent comprising syngas; and
a reactor unit configured to receive the cathode effluent comprising syngas, thereby producing a chemical product or a fuel-based product.

17. The system according to claim 16, further comprising:
a carbon dioxide source for passing the reactor unit;
wherein the reactor unit is further configured to receive a carbon dioxide feed stream from the carbon dioxide source with the cathode effluent comprising syngas, thereby producing the chemical product or the fuel-based product.

18. The system according to claim 16, further comprising:
a second electrolyzer comprising a cathode, an anode and an electrolyte inserted between the cathode and the anode, wherein the second electrolyzer is configured to receive in the cathode a second steam feed stream, thereby producing a cathode effluent comprising hydrogen;
wherein the reactor unit is further configured to receive the cathode effluent from the second electrolyzer with the cathode effluent from the first electrolyzer for producing the chemical product or the fuel-based product.

19. The system according to claim 16, further comprising:
a second electrolyzer comprising a cathode, an anode and an electrolyte inserted between the cathode and the anode, wherein the second electrolyzer is configured to receive in the cathode a steam and carbon dioxide feed stream, thereby producing a cathode effluent comprising syngas;
wherein the reactor unit is further configured to receive the cathode effluent from the second electrolyzer with the cathode effluent from the first electrolyzer for producing the chemical product or the fuel-based product.

20. The system according to claim 16, wherein the reactor unit is one of a Fischer-Tropsch reactor unit or a methanol and dimethyl ether reactor unit.
